(19) United States
(12) Patent Application Publication
(10) Pub. No.: US 2017/0239273 A1
(43) Pub. Date: Aug. 24, 2017

(54) COMPOSITIONS AND METHODS TO TREAT AND/OR PREVENT VISION DISORDERS OF THE LENS OF THE EYE

(71) Applicants: Kang ZHANG, San Diego, CA (US); Stanley KIM, San Diego, CA (US); Jie ZHU, San Diego, CA (US); Rui HOU, Guangzhou, Guangdong (CN); J Jefferson PERRY, Carlsbad, CA (US)

(72) Inventors: Kang ZHANG, San Diego, CA (US); Stanley KIM, San Diego, CA (US); Jie ZHU, San Diego, CA (US); Rui HOU, Guangzhou, Guangdong (CN); J Jefferson PERRY, Carlsbad, CA (US)

(21) Appl. No.: 15/505,540

(22) PCT Filed: Aug. 22, 2015
(86) PCT No.: PCT/US15/46437

§ 371 Date: Feb. 21, 2017

Related U.S. Application Data
(60) Provisional application No. 62/040,721, filed on Aug. 22, 2014.
(60) Provisional application No. 62/195,139, filed on Jul. 21, 2015.

(51) Int. Cl. A61K 31/575; A61K 9/00; A61K 47/40

(52) U.S. Cl. CPC A61K 31/575; A61K 47/40; A61K 9/0048

(57) ABSTRACT

The disclosure generally relates to compositions and uses thereof to treat vision disorders that affect the normal function of the lens in the eye in a subject having or at risk of developing such vision disorders.

### CROSS REFERENCE TO RELATED APPLICATIONS

This application claims priority from U.S. Provisional Application Number 62/040721 filed on Aug. 22, 2014, which is hereby incorporated herein by reference in its entirety.

### FIELD OF THE INVENTION

The disclosure generally relates to compounds and uses thereof to treat vision disorders that affect the normal function of the lens in the eye in a subject having or at risk of developing such vision disorders.

### SUMMARY OF THE INVENTION

The invention provides a method of treating or preventing vision disorders, the method comprising administering to an individual in need thereof an effective amount of a composition comprising a compound of formula I or formula II: formula I having a structure of:

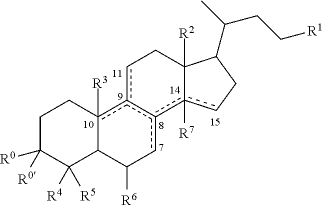

wherein:

R0 and R0′ is hydroxyl, -—OSO3H, —OSO3-, —OCOCH3, —OPO3H, —OPO3-, or hydrogen, or R0 and R0’ together represent a carbonyl group;
R¹ is

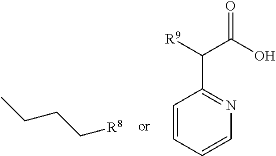

R², R³, R⁴, R⁵, R⁷ are each H or Me;
R⁶ is H or Me or OH or oxo (═O) or halide;
R⁸ is a linear or branched alkyl, aryl, alkene, alkyne, a substituted alkene, a substituted alkyl, a substituted alkyne, a substituted aryl, an alkyl halide, alkoxy such as an alcohol or an aryloxy, or an acetyl or ester group having from 2 to 6 carbon;
R¹ is at carbon 16 or carbon 17, at least one of the dashed lines between carbons 7 and 8, carbons 8 and 9, carbons 9 and 10, carbons 9 and 11, carbons 8 and 14, or carbons 14 and 15 indicates a double bond, with the proviso that there be no adjacent double bonds on a ring or adjacent rings (e.g., if a double bond is present between carbons 8 and 9, no other double bonds are present in either of the two adjacent rings, or double bonds are not co-present between carbons 8 and 14 and carbons 14 and 15), and/or R³ is H if a double bond is present between carbons 9 and 10 and/or R⁷ is H if a double bond is present between carbons 8 and 14 or carbons 14 and 15; and formula II having a formula as:

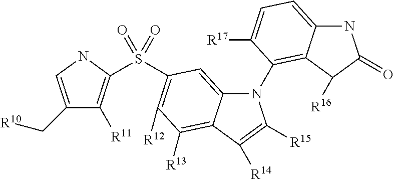

a prodrug or pharmaceutically acceptable salt thereof.

The invention also provides an ophthalmic pharmaceutical composition comprising a pharmaceutically acceptable ophthalmic carrier and a compound of formula I or formula II.

In various aspects of the method and/or composition, the compound of formula I has a structure of formula IA:

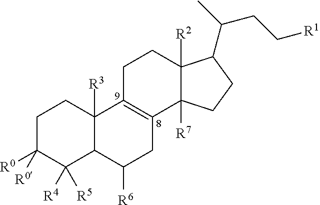

wherein:
R0 and R0′ is hydroxyl, —OSO3H, —OSO3-, —OCOCH3, —OPO3H, —OPO03-, or hydrogen, or R0 and R0′ together represent a carbonyl group;
R¹ is a linear or branched alkyl, aryl, alkene, alkyne, a substituted alkene, a substituted alkyl, a substituted alkyne, a substituted aryl, an alkyl halide, alkoxy such as an alcohol or an aryloxy, or an acetyl or ester group having from 2 to 6 carbon;
R², R³, R⁴, R⁵, R⁷ are each H or Me;
R⁶ is H or Me or OH or oxo (═O) or halide;
a prodrug or pharmaceutically acceptable salt thereof.

In various aspects of the method, the vision disorder is a disorder of the eye that affects function, clarity and/or structure of the lens of the eye. Such eye diseases include, but are not limited to, cataracts of the eye, presbyopia of the eye, and nuclear sclerosis of the eye lens. In addition, vision disorders refer to retinal degeneration, such as as Refsum disease, Smith-Lemli-Opitz syndrome (SLOS) and Schnyder crystalline corneal dystrophy (SCCD), abetalipoproteinemia and familial hypobetalipoproteinemia.

One embodiment of the invention provides a method of ameliorating at least one symptom associated with a vision disorder by administering to a subject a therapeutically or prophylactically effective amount of a sterol of formula 1. In various aspects of the method, the composition is administered topically, subconjunctivally, retrobulbarly, periocularly, subretinally, suprachoroidally, or intraocularly. Subjects that receive the invention sterol can include, but are not limited to mammals, avians, amphibians, reptiles and other vertebrates. In some embodiments, the subjects are horses, pigs, dogs, cats, rodents and/or other companion pets. In other embodiments, the subjects are humans.

Some embodiments of the invention relate to an ophthalmic pharmaceutical composition comprising the invention sterol in an ophthalmic pharmaceutically acceptable carrier. In some embodiments, the pharmaceutical composition comprises cholesterol precursors, e.g., sterold, thereof in an ophthalmic pharmaceutically acceptable carrier. In certain embodiments of the invention, the pharmaceutically acceptable carrier is water, a buffer or a solution of sodium chloride. In some embodiments, the pharmaceutically acceptable carrier is sterile. In other embodiments, the pharmaceutically carrier is an ointment. In still other embodiments, the pharmaceutically acceptable carrier is a gel. Gels can be formulated using gel formulating materials that are well known in the art, including but not limited to, high viscosity carboxymethylcellulose, hydroxypropylmethylcellulose, polyethylene oxide and carbomer. In some aspects of the composition, the pharmaceutically acceptable ophthalmic carrier is a cyclodextrin. In a specific aspect, the cyclodextrin is (2-hydroxypropyl)-β-cyclodextrin.

Certain embodiments of the invention also contemplate kits that comprise components useful for treating and/or preventing a symptom associated with a vision disorder. Such kits comprise a container comprising invention sterol in a pharmaceutically acceptable carrier and instructions for administering the invention sterol such that at least one symptom associated with the vision disorder is ameliorated or prevented. Such vision disorder include, but are not limited to, cataracts, presbyopia, and nuclear sclerosis of the eye lens. In addition, vision disorders refer to retinal degeneration, such as as Refsum disease, Smith-Lemli-Opitz syndrome (SLOS) and Schnyder crystalline corneal dystrophy (SCCD), abetalipoproteinemia and familial hypobetalipoproteinemia. The containers included in some of the kits contemplated herein are droppers for the administration of eye drops. In other embodiments, the container is a tube for dispensing ointment or gel. In still other embodiments, the container is any appropriate container for drug delivery including, but not limited to, a syringe, or other container appropriate for delivery of a drug ophthalmically or topical application.

In other aspects, the invention provides a method for inhibiting or preventing protein aggregation. In various aspects of the method, the protein is an amyloid-forming protein or a protein underlying a loss-of-function disease. In some aspects, the amyloid-forming protein is selected from the group consisting of Hsp27, αA-crystallin, αB-crystallin, βB2-crystallin, βB1-crystallin, γD-crystallin, Hsp22, Hsp20, tau, Alpha-synuclein, IAPP, beta-amyloid, PrP, Huntingtin, Calcitonin, Atrial natriuretic factor, Apolipoprotein AI, Serum amyloid A, Medin, Prolactin, Transthyretin, Lysozyme, Beta 2 microglobulin, Gelsolin, Keratoepithelin, Cystatin, Immunoglobulin light chain AL, and S-IBM. In other aspects, the protein underlying a loss-of-function disease is selected from the group consisting of mutant β-glucosidase, cystic fibrosis transmembrane receptor, hexosaminidase A, hexosaminidase B, β-galactosidase, and alpha-glucosidase.

Other features and advantages of the present disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description. The entire document is intended to be related as a unified disclosure, and it should be understood that all combinations of features described herein are contemplated, even if the combination of features are not found together in the same sentence, or paragraph, or section of this document. In addition to the foregoing, the invention includes, as an additional aspect, all embodiments of the invention narrower in scope in any way than the variations specifically mentioned above. For example, if aspects of the invention are described as “comprising” a feature, embodiments also are contemplated “consisting of” or “consisting essentially of” the feature.

### BRIEF DESCRIPTION OF THE DRAWINGS

FIG. 1. Lanosterol significantly reduced intracellular aggregation of various cataract-causing crystallin mutants in a dose-dependent manner

FIG. 2. Cholesterol did not affect the formation of intracellular aggregates of various cataract-causing crystallin mutants

FIG. 3. Lanosterol significantly reduced intracellular aggregation of various cataract-causing crystallin mutants

FIG. 4. Lanosterol but not cholesterol increased the amounts of soluble proteins. The crystallins were detected by anti-GFP antibody. Beta-actin was used as an internal loading control. TCL: total cell lysates; S: supernatant; P: precipitation. Treatment by lanosterol, but not cholesterol, increased cataract-causing mutant crystallins in soluble fractions when compared to a control group or a mutant LSS group. a, Human lens progenitor cells were transfected with mutant crystallin genes for 4 h, and then incubated in fresh culture medium for another 24 h. The cells were harvested and lysed. Supernatant and insoluble fractions were separated by centrifugation and analysed by western blot analysis. LSS and crystallin fusion proteins were identified by antibodies against Flag and GFP tags, respectively. The lanosterol-treated group is highlighted by red boxes. Cells treated with 1% DMSO were used as a control. b-Actin was used as an internal protein loading control of total cell lysates (TCL). S, supernatant; P, insoluble fraction.

FIG. 5. In vitro protein assay indicated that lanosterol could dissociate the aggregates of various WT and mutated crystallins, while cholesterol could not. Lanosterol increased the soluble fractions of various crystallin mutants in human lens progenitor cells.

FIG. 6 In vitro protein assay indicated that lanosterol could dissociate the aggregates of various WT and mutated crystallins, while cholesterol could not. Evaluation of the effect of lanosterol on the dissolution of crystallin aggregates by turbidity. Crystallin aggregates were formed by incubating 5 mgml21 protein solution at 60° C. for 2 h (α-crystallins) or 37° C. for 48 h (β- and γ-crystallins) in the presence of 1M guanidine chloride. The preformed aggregates were re-suspended in PBS at a final protein concentration of 0.2 mg/ml and were treated with 500 μM sterols in 500 μM DPPC liposome and incubated at 37° C. for 24 h. Aggregates treated with 500 μM DPPC liposome only were used as the controls.

FIG. 7. In vitro protein assay indicated that lanosterol could dissociate the aggregates of various WT and mutated crystallins, while cholesterol could not.

Lanosterol but not cholesterol can dissociate the preformed amyloid-like fibrils/aggregates of various WT and mutated crystallins.

The amounts of soluble proteins after treating the crystallin aggregates with lanosterol but not cholesterol. The α-crystallin aggregates were formed by incubating 5 mg/ml protein solutions at 60° C. for 48 h in the presence of 1 M guanidine chloride. The β- and γ-crystallin aggregates were prepared by incubating the protein solutions at 37° C. and pH 3 for 48 h. The preformed aggregates were re-suspended in PBS with a final protein concentration of 0.1 mg/ml. The re-suspended aggregates were treated by 50 μM sterols in 50 μM DPPS liposome and incubated at 37° C. for 144 h. The aggregates treated by 50 μM DPPS liposome were used as the controls. The supernatant and precipitation fractions were separated by centrifugation. The protein concentrations in the supernatant fraction were determined by absorbance at 280 nm and the percentages of the soluble proteins were calculated from three independent repetitions.

Representative negatively stained EM pictures of the aggregates of αB-crystallin R120G mutant treated by liposome, lanosterol in liposome and cholesterol in liposome, respectively. The EM samples were stained by 1.25% uranyl acetate.

Lanosterol but not cholesterol dissociates the aggregates formed by αB-crystallin R120G mutant. Quantitative analysis was performed by calculating the number of particles with length over 50 nm from six randomly picked viewing field. Each viewing field contained 30-50 particles.

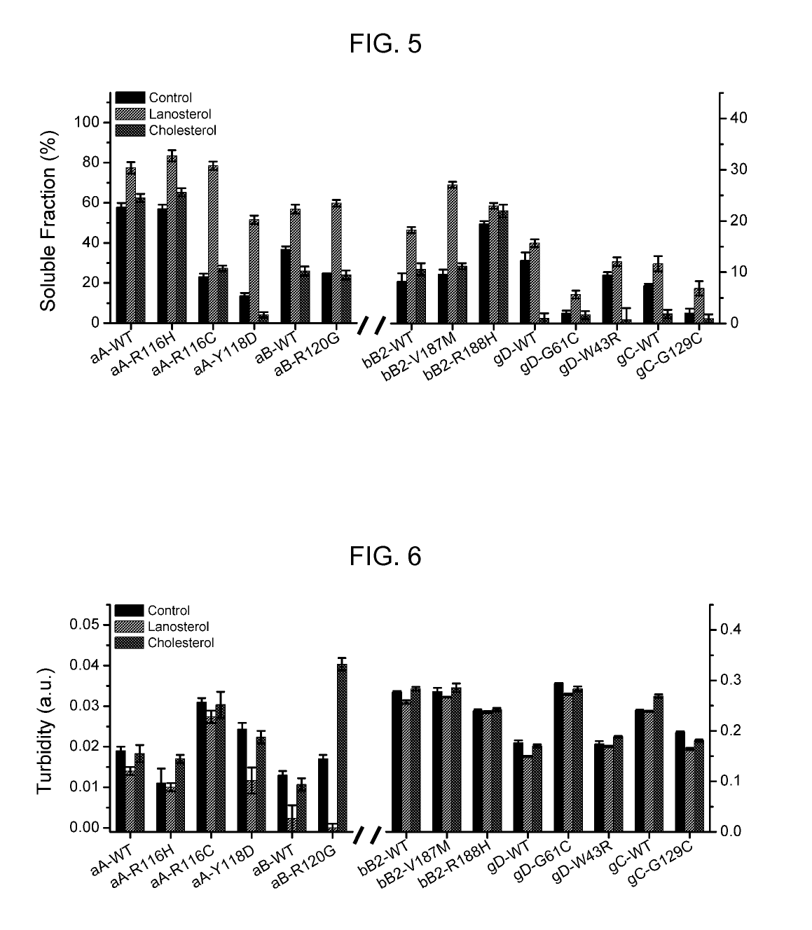

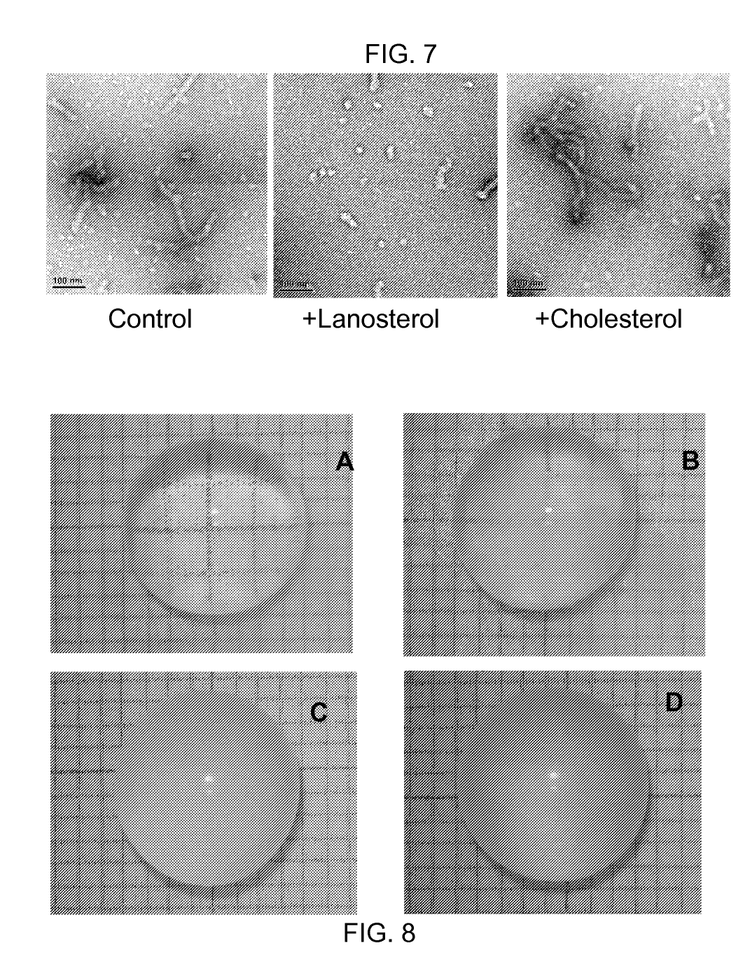

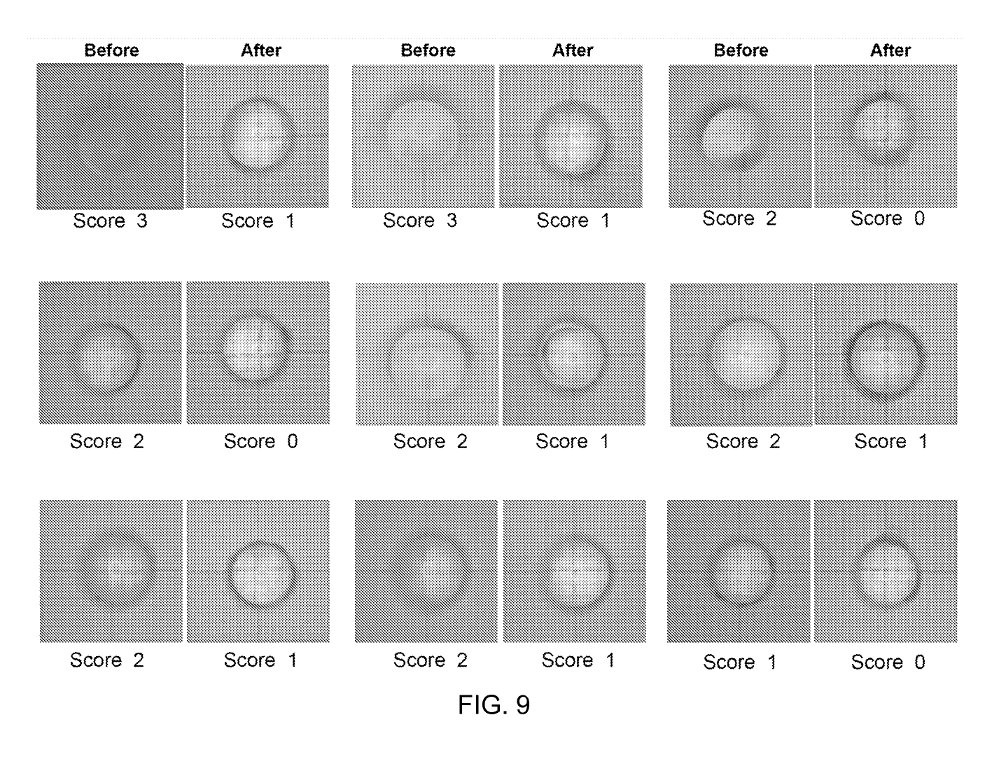

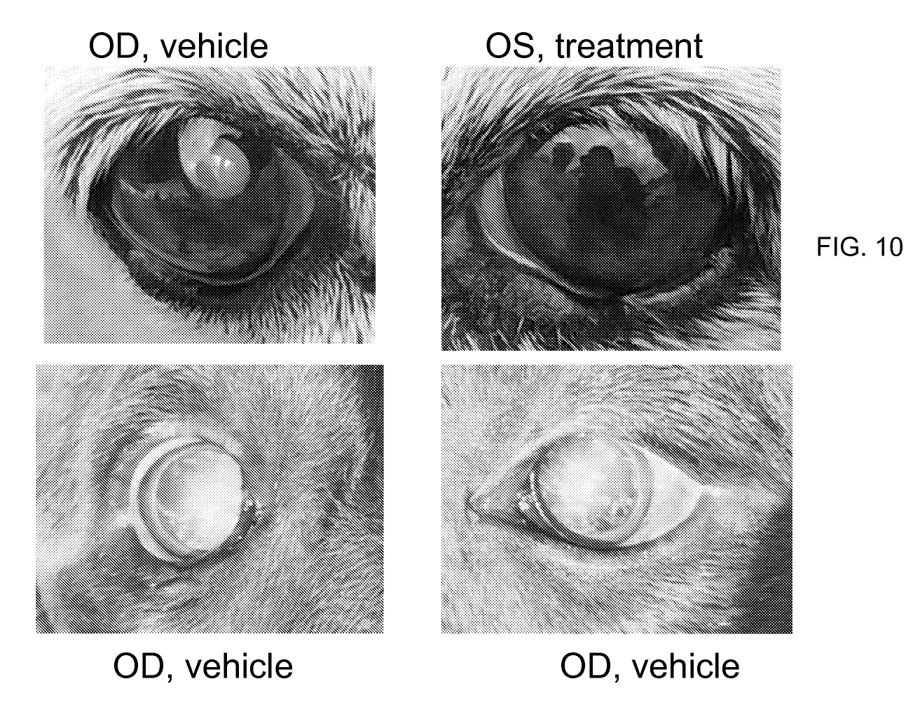

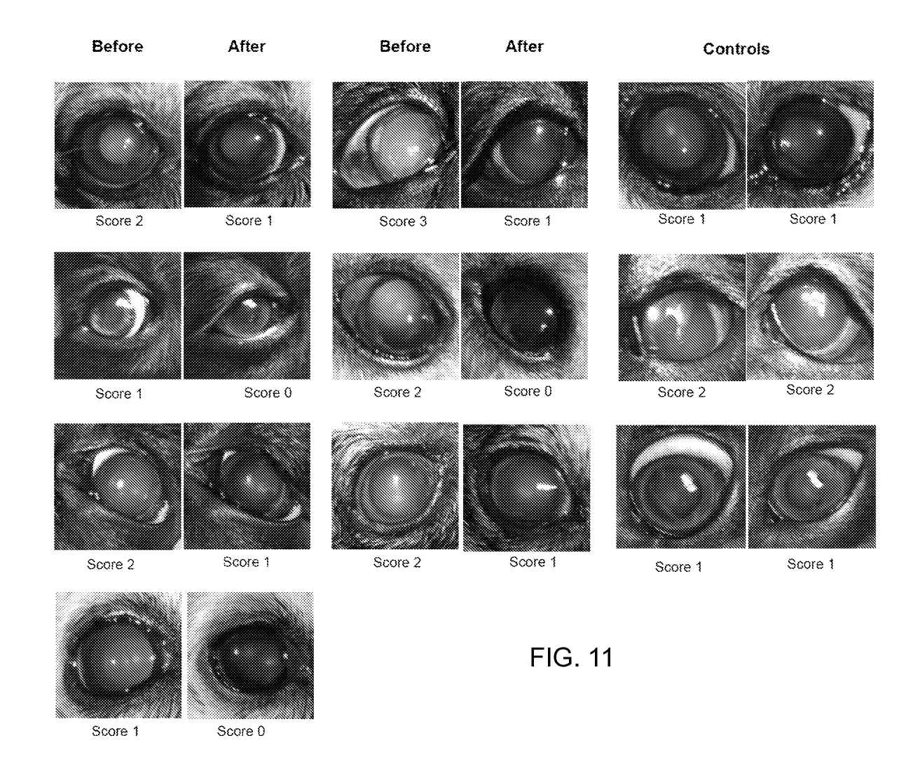

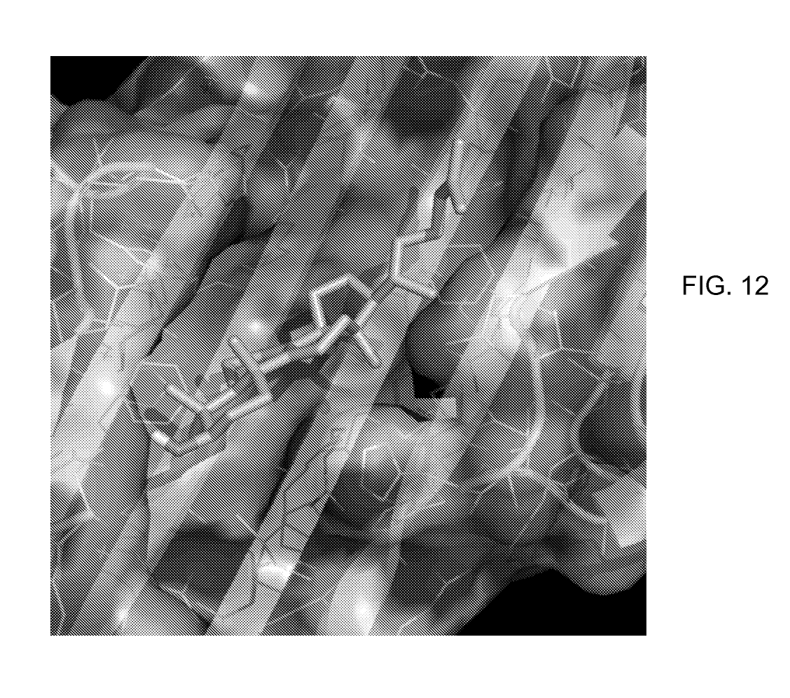

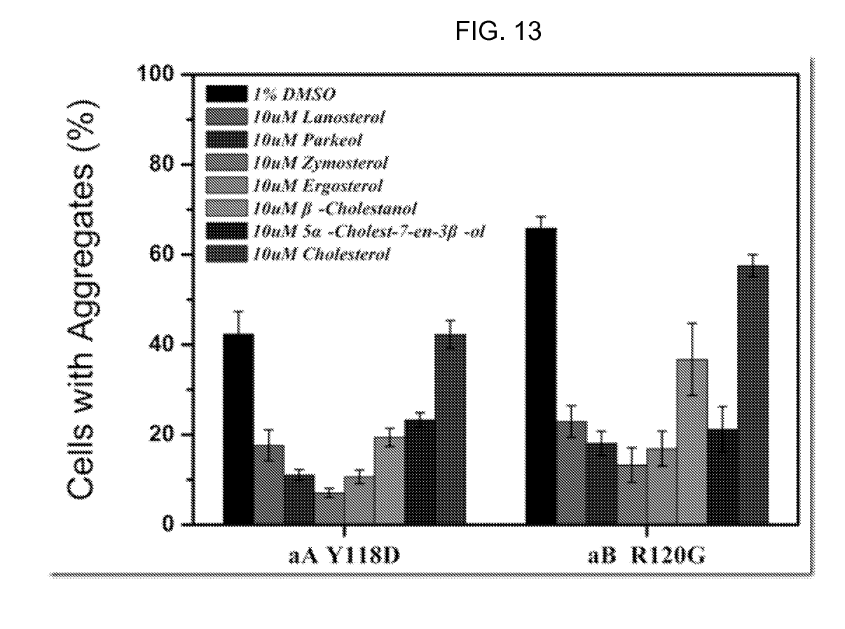

FIG. 8. Lanosterol reduced cataract severity and increased clarity. Photographs of a cataractous rabbit lens treated with lanosterol showing increased lens clarity. FIG. 5A. Grading of rabbit lens A. clear; B. 1+cloudy; C. 2+cloudy; D. 3+cloudy

FIG. 9. Lanosterol reduced cataract severity and increased clarity in isolated cataractous rabbit lenses. Rabbit lenses (n=13) were dissected and incubated with lanosterol for 6 days and subsequently assessed for lens clarity and transparency. Pairs of photographs of each cataractous rabbit lens showing before and after treatment with scores underneath are shown.

FIG. 10. Treatment of cataract in dogs reduced cataract.

FIG. 11. Lanosterol reduced cataract severity and increased lens clarity in dogs. Dog eyes with cataracts (n=7) were treated with lanosterol for 6 weeks and assessed for lens clarity and transparency. A pair of photographs of each study eye before and after treatment is shown with scores underneath. Three control eyes treated with vehicles alone are also presented.

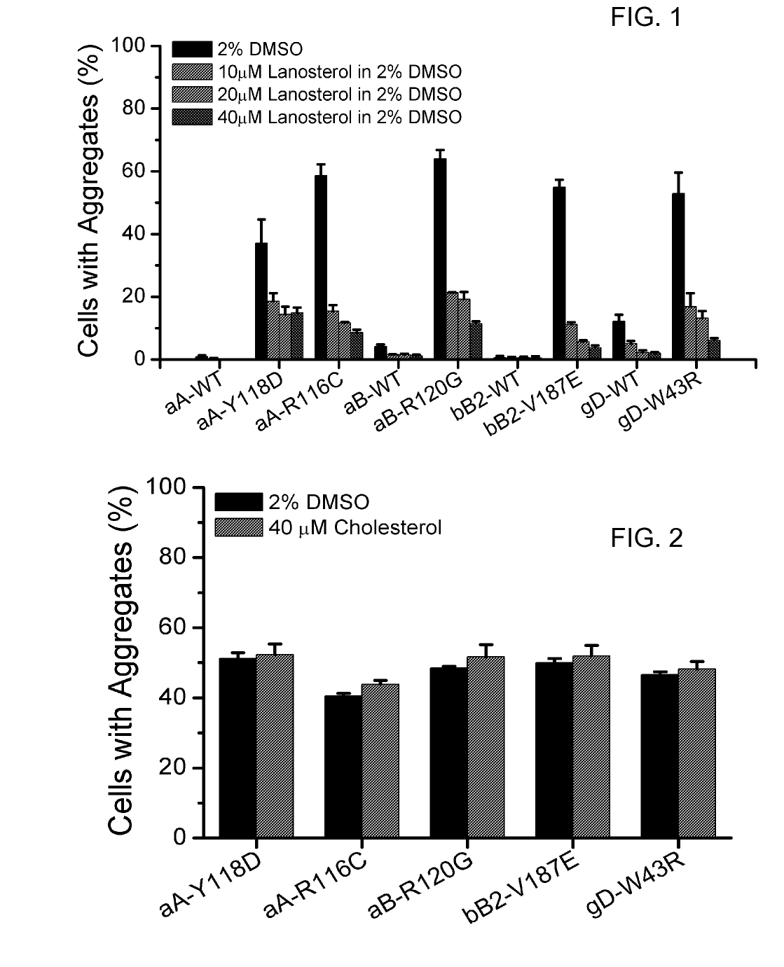

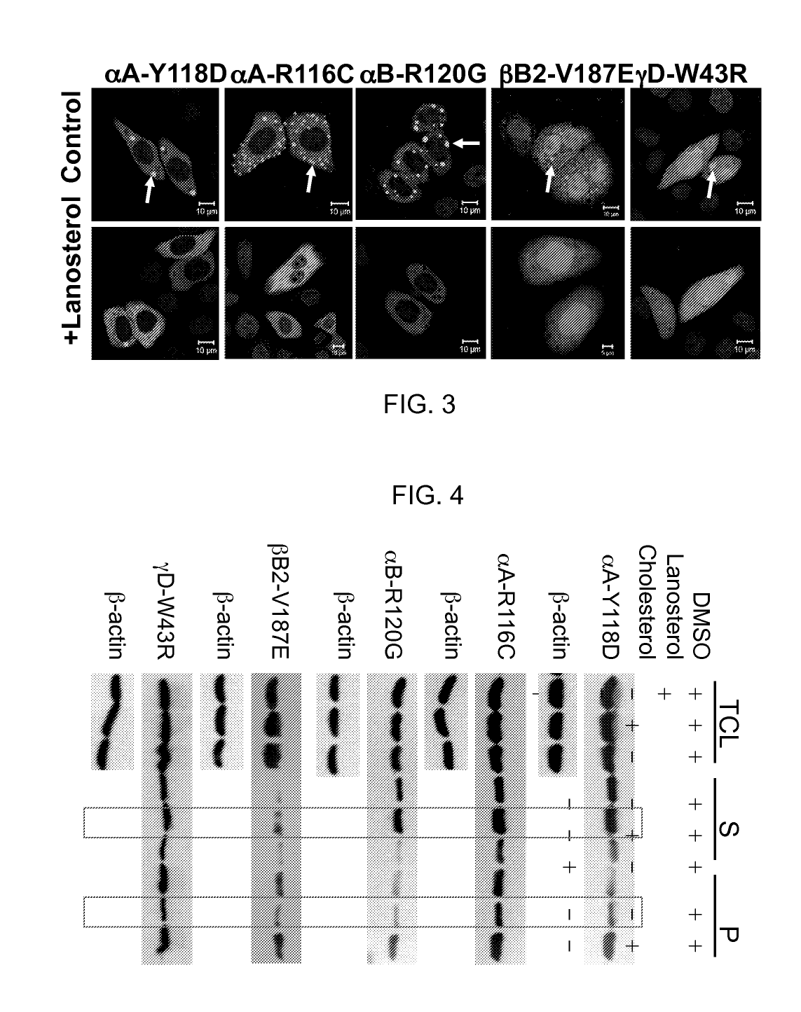

FIG. 12. FIG. 1. Lanosterol bound in the central pocket of the human alphaB crystallin crystal structure (protein databank code: 2WJ7)

FIG. 13. Two mutant crystal proteins were introduced into cells. The accumulated crystal protein were treated by adding different agents. Compared with 1% DMSO solution, addition of 10 μM lanosterol, 10 μM Parkeol, 10 μM Zymosterol, 10 μM ergosterol, 10 μMβ-cholestanol and 10 μM 5α-cholest-7-en-3β-ol caused significant dissolving of the accumulated crystal protein.

### DETAILED DESCRIPTION

Reference will now be made in detail to specific embodiments of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims. In the following description, specific details are set forth in order to provide a thorough understanding of the present invention. The present invention may be practiced without some or all of these specific details. In addition, well-known features may not have been described in detail to avoid unnecessarily obscuring the invention.

The present invention relates to a method of and compositions for treating or preventing vision disorders that affect the normal structure of the eye in a subject having or at risk of developing such vision disorders comprising administering to such subject a composition comprising a pharmaceutically acceptable carrier and a pharmaceutically effective amount of a sterol having the formula I or formula II. For example, an exemplary compound of the invention comprises administering to a patient an opthalmological pharmaceutically effective amount of a cholesterol precursor. In other embodiments, the compound is a cholesterol intermediate in the cholesterol biosynthesis. Cholesterol intermediates include parkeol, zymosterol (5α-Cholesta-8,24-dien-3β-ol), and ergosterol.

In other embodiments, the present disclosure describes sterols and methods of using sterols. For example, the sterols of formula 1 are formulated in ophthalmic pharmaceutical compositions comprising a pharmaceutically acceptable ophthalmic carrier to inhibit crystallin protein aggregation. In certain other embodiments, the present disclosure describes methods of using sterols of formula 1 to inhibit crystallin protein aggregation. In yet other embodiments, compounds of the invention are able to reverse aggregation of crystallin protein and inhibit further aggregation of crystallin protein.

Methods of Treating or Preventing Vision Disorders

The present invention provides ophthalmic pharmaceutical compositions and methods of using the present invention in preventing and/or treating vision disorders that affect the normal structure of the lens in the eye in a subject having or at risk of developing such vision disorders. As described herein, a vision disorder that affects the normal structure of the lens in the eye (referred herein as the phrase “vision disorder”) refers to conditions that affect the structure of the lens as to cause vision dysfunction, such as changes to the clarity or rigidity of the lens of the eye. Such conditions include cataracts, presbyopia and nuclear sclerosis. In addition, vision disorders refer to retinal degeneration, such as as Refsum disease, Smith-Lemli-Opitz syndrome (SLOS) and Schnyder crystalline corneal dystrophy (SCCD), abetalipoproteinemia and familial hypobetalipoproteinemia. In certain embodiments, the present invention provides compositions and methods of use thereof to alleviate or reverse crystalline protein aggregation. In alternative embodiments, there are provided compositions and methods for inhibiting, preventing and/or treating the disruption of intra- or inter-protein interactions that form the macro-structure essential for lens transparency and refractive index.

The term “cataract” as referred to in the present invention means a disease or condition that exhibits symptoms of causing cloudiness or opacity on the surface and/or the inside of the lens or inducing the swelling of the lens, and it includes both congenital cataract and acquired cataract (cf. PDR Staff, “PDR of Ophthalmic Medicines 2013”, PDR Network, 2012). In some embodiments, the cataract is an age-related cataract, a diabetic cataract, a cataract associated with surgery, a cataract resulting from exposure to radiation, a cataract resulting from a genetic illness, a cataract resulting from an infection, or a cataract resulting from medication. In some embodiments, the individual has a hereditary form of cataract with early onset. Concrete examples of such are congenital cataract such as congenital pseudo-cataract, congenital membrane cataract, congenital coronary cataract, congenital lamellar cataract, congenital punctuate cataract, and congenital filamentary cataract; and acquired cataract such as geriatric cataract, secondary cataract, browning cataract, complicated cataract, diabetic cataract, traumatic cataract, and others inducible by electric shock, radiation, ultrasonic, drugs, systemic diseases, and nutritional disorders. Acquired cataract further includes postoperative cataract with symptoms of causing cloudiness in the posterior encapsulating a lens inserted to treat cataract.

Nuclear sclerosis refers to a condition, generally in older animals, that results similarly in opacity of the lens. It is an age-related change in the density of the crystalline lens nucleus that is caused by compression of older lens fibers in the nucleus by new fiber formation.

Presbyopia refers to a vision condition in which the crystalline lens of the eye loses its flexibility, which makes it difficult to focus on close objects.

In some embodiments, the invention provides a method of treating or preventing a vision disorder, the method comprising administering to an individual in need thereof an effective amount of a composition comprising a compound having a structural formula I. In some embodiments, the compound is a sterol having a structural formula I.

An individual “in need of” treatment according to the invention is an individual that is suffering from a vision disorder that affects the normal function of the lens in the eye. For example, the individual may have or is at risk for developing an age-related cataract or a cataract. Individuals at risk of developing a cataract include, but are not limited to, individuals with a family history of developing cataracts, individuals with a mutation linked to a cataract, individuals exposed to radiation, diabetics, and the like. For example, in one aspect, the individual has been diagnosed with cataract in one eye, and the compound is administered to prevent or slow cataract formation in the contralateral eye. Similarly, an individual “in need of” treatment according to the invention is an individual that may have or is at risk for developing presbyopia. Similarly, an individual “in need of ” treatment according to the invention is an individual that has or is at risk for developing nuclear sclerosis. Preferably the individual is human, however, animals that suffer from or who are at risk for an eye disease (animals in need of treatment) can also be identified by one skilled in the art. Mammals in need of treatment, such as cats, dogs, pigs, horses, cows and rodents can be identified. Additionally, animals such as avians, reptiles, amphibians, and fish that are in need of treatment can be identified.

“Treating” a vision disorder does not require a 100% abolition or reversal of a vision disorder. In some embodiments, “treating” vision disorders according to inventive method alleviates, inhibits, prevents and/or reverses dysfunction of the lens, e.g., opacity or inflexibility of the lens by, e.g., at least about 5%, at least about 10% or at least about 20% compared to levels observed in the absence of the inventive composition or method (e.g., in a biologically-matched control subject or specimen that is not exposed to the invention composition or compound of the inventive method). In some embodiments, dysfunction (such as cataract formation, opacity or crystalline aggregation on or in the lens) is treated by at least about 30%, at least about 40%, at least about 50%, or at least about 60%, at least about 70%, at least about 80%, at least about 90%, or more (about 100%) compared to lens dysfunction in the absence of the compound of the inventive method. Lens dysfunction, such as opacity or cloudiness or cataracts, generally are detected using any of a number of optic tests including, but not limited to, visual acuity testing, ophthalmoscopy, slit-lamp examination, keratometry, tonometry, contrast testing, glare sensitivity, wavefront mapping.

Similarly, “prevention” does not require 100% inhibition or deterrence of a vision disorder. For example, any reduction in cloudiness or opacity, or deceleration of cataract progression constitutes a beneficial biological effect in a subject. Also exemplary, any decrease in crystalline aggregation in the lens of an eye constitutes a beneficial biological effect. In this regard, the invention reduces the vision disorder, e.g., at least about 5%, at least about 10% or at least about 20% compared to levels observed in the absence of the inventive method (e.g., in a biologically-matched control subject or specimen that is not exposed to the compound of the inventive method). In some embodiments, the vision disorder is reduced by at least about 30%, at least about 40%, at least about 50%, or at least about 60%, at least about 70%, at least about 80%, at least about 90%, or more (about 100%).

Inhibiting, preventing or reversal of dysfunction does not require a 100% inhibition, prevention, abolition or reversal. For example, any inhibition of aggregation constitutes a beneficial biological effect in a subject. In this regard, the invention inhibits a vision disorder that affects the normal function of the lens of the eye in a subject, e.g., at least about 5%, at least about 10% or at least about 20% compared to levels observed in the absence of the inventive method (e.g., in a biologically-matched control subject or specimen that is not exposed to the compound of the inventive method). In some embodiments, the vision disorder is inhibited, prevented and/or reversed by at least about 30%, at least about 40%, at least about 50%, or at least about 60%. In some embodiments, the inventive method inhibits amyloid formation by at least about 70%, at least about 80%, at least about 90%, or more (about 100%) compared to amyloid formation in the absence of the compound of the inventive method.

An “effective amount” of an ophthalmic pharmaceutical composition comprising a compound of formula 1 is an amount that inhibits, prevents or reverses dysfunction of the lens in an individual. An ophthalmic pharmaceutical composition of the present invention is being administered to a subject in need thereof at an effective amount to treat the vision disorder. As used herein, “therapeutically effective amount” means a dose that alleviates at least one of the signs, symptoms, or causes of a vision disorder, or any other desired alteration of a biological system. In preventative applications, the term “prophylactically effective amount” means a dose administered to a patient susceptible to or otherwise at risk of a particular disease, which may be the same or different dose as a therapeutically effective amount. The effective amount of the composition for a particular individual can depend on the individual, the severity of the condition of the individual, the type of formulation being applied, the frequency of administration, and the duration of the treatment. In accordance with the present invention, administration of an ophthalmic pharmaceutical formulation of the present invention such as, e.g., sterol, even at relatively low concentrations in liquid drops, e.g., at least 10-9 M, at least 0.5 to 1×10-8 M, at least 0.5 to 1×10-7 M, at least 0.5 to 1×10-6 M, at least 0.5 to 1×10-5 M, at least 0.5 to 1×10-4 M, or at least 0.5 to 1×10-3 M, or any concentration falling in a range between these values (e.g., 10-9 M to 10-3 M), may reverse such vision disorders with only one, two, three or multiple, daily applications and does so rapidly.

### Route of Administration

As will be understood by those skilled in the art, the most appropriate method of administering a compound to a subject is dependent on a number of factors, for example, the compound according to the invention is administered locally to the eye, e.g., topically, subconjunctivally, retrobulbarly, periocularly, subretinally, suprachoroidally, or intraocularly.

Pharmaceutical compositions that are particularly useful for administration directly to the eye include aqueous solutions and/or suspensions formulated as eye drops and thickened solutions and/or suspensions formulated as ophthalmic gels (including gel-forming solutions) or ointments, which is an ophthalmic solution, ophthalmic ointment, ophthalmic wash, intraocular infusion solution, wash for anterior chamber, internal medicine, injection, or preservative for extracted cornea. Other dosage forms for ophthalmic drug deliver include ocular inserts, intravitreal injections and implants. Injectable solutions can be directly injected into the cornea, crystalline lens and vitreous or their adjacent tissues using a fine needle. The composition also can be administered as an intraocular perfusate.

Additional contemplated routes of administration include, but are not limited to, one or more of: oral (e.g., as a tablet, capsule, or as an ingestible solution), mucosal (e.g., as a nasal spray or aerosol for inhalation), nasal, parenteral (e.g., by an injectable form), gastrointestinal, intraspinal, intraperitoneal, intramuscular, intravenous, intrauterine, intradermal, intracranial, intratracheal, intravaginal, intracerebroventricular, intracerebral, subcutaneous, transdermal, rectal, buccal, epidural and sublingual.

In some embodiments, the mode for delivery of a composition of the invention to the eye is via a contact lens. The lens may be provided pre-treated with the desired compound. Alternatively, the lens is provided in a kit with components for preparing a coated lens, which are provided as lyophilized powders for reconstitution or as concentrated or ready-to-use solutions. The compositions can be provided as kits for single or multi-use.

In some embodiments, the mode for delivery of a composition of the invention to the eye is via an ophthalmic rod (Gwon et al., Ophthalmology. 1986 September; 93(9 Suppl):82-5). In some embodiments, the mode for delivery of a composition of the invention to the eye is via an intraocular lens-hydrogel assembly (Garty et al., Invest Ophthalmol Vis Sci, 2011 Aug. 3; 52(9):6109-16).

### Dose

The composition comprising the compound is provided in a therapeutically effective amount that achieves a desired biological effect at a medically-acceptable level of toxicity. The dosage of the compositions may vary depending on the route of administration and the severity of the disease. The dosage may also be adjusted depending on the body weight, age, sex, and/or degree of symptoms of each patient to be treated. The precise dose and route of administration will ultimately be at the discretion of the attendant physician or veterinarian. It will be appreciated that it may be necessary to make routine variations to the dosage depending on the age and weight of the patient as well as the severity of the condition to be treated. The frequency of administration depends on the formulation and the aforementioned parameters. For example, it may be desirable to apply eye drops at least once per day, including 2, 3, 4, or 5 times per day.

Persons of ordinary skill can easily determine optimum dosages, dosing methodologies and repetition rates. Optimum dosages may vary depending on the relative potency of the particular pharmaceutical composition and the method of administration. Acceptable dosages can generally be estimated based on EC50 (effective concentration for 50% of the test group) found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the therapeutic compositions described herein are administered in maintenance doses, ranging from 0.01 μg to 100 g per kg of body weight, once or more daily, to once every 20 years. Exemplary doses of the compounds for administration to a human (of approximately 70 kg body weight) via systemic route are 0.1 mg to 5 g, e.g., 1 mg to 2.5 g of the compound per unit dose.

Preferred concentrations of the compound of formula I, IA, IB, or II, or a compound listed in Table 1, range from about 1 μg/ml to 500 μg/ml, for example, about 1 μg/ml, about 2 μg/ml, about 3 μg/ml, about 4 μg/ml, about 5 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 120 μg/ml, about 140 μg/ml, about 160 μg/ml, about 180 μg/ml, about 200 μg/ml, about 250 μg/ml, about 300 μg/ml, about 350 μg/ml, about 400 μg/ml, about 450 μg/ml, or about 500 μg/ml. The inhibitor may be provided in combination with other pharmaceutically active agents.

The pharmaceutical compositions described herein can be administered as a single dose or in multiple doses; administered either as individual therapeutic agents or in combination with other therapeutic agents; and combined with conventional therapies, which may be administered sequentially or simultaneously. In one embodiment of the invention, daily dosages in human and/or animal therapy of the present ophthalmic formulations are about 1 drop per eye, about 2 drops per eye, about 3 drops per eye, about 4 drops per eye, about 5 drops per eye, about 6 drops per eye, about 7 drops per eye, about 8 drops per eye, about 9 drops per eye, about 10 drops per eye, about 11 drops per eye, about 12 drops per eye or more than about 12 drops per eye. In another embodiment of the invention, daily administration schedule for the present ophthalmic formulations in human and/or animal therapy is about 1 time per day, about 2 times per day, about 3 times per day, about 4 times per day, about 5 times per day, about 6 times per day, about 7 times per day, about 8 times per day, about 9 times per day, about 10 times per day, about 11 times per day, about 12 times per day or more than about 12 times per day. Dosages can be standardized for instance by means of a standard pharmacopeial medicinal dropper of 3 mm in external diameter, which when held vertically delivers 20 drops of water of total weight of 0.9 to 1.1 grams at 25° C.

When administered according to the dosage schedule described above, the treatment regimen in humans and/or animals can continue indefinitely or until no further improvement is observed. Alternately, the treatment regimen can last for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 60 days, 70 days, 80 days, 90 days, 100 days, 150 days, 200 days, 250 days, 300 days, 400 days, 500 days, 750 days, 1000 days or more than 1000 days.

### Compounds Effective in Treating or Preventing Cataract

In various embodiments, the compound of the inventive method or composition is a compound of formula I or formula II: formula I having a structure of:

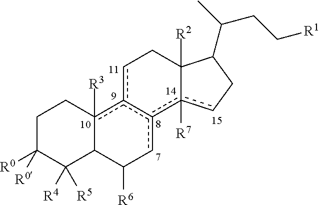

wherein:
R0 and R0′ is hydroxyl, —OSO3H, —OSO3-, —OCOCH3, —OPO3H, —OPO3-, or hydrogen, or R0 and R0’ together represent a carbonyl group;
R¹ is

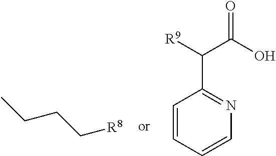

R², R³, R⁴, R⁵, R⁷ are each H or Me;
R⁶ is H or Me or OH or oxo (═O) or halide;
R⁸ is a linear or branched alkyl, aryl, alkene, alkyne, a substituted alkene, a substituted alkyl, a substituted alkyne, a substituted aryl, an alkyl halide, alkoxy such as an alcohol or an aryloxy, or an acetyl or ester group having from 2 to 6 carbon;
R¹ is at carbon 16 or carbon 17, at least one of the dashed lines between carbons 7 and 8, carbons 8 and 9, carbons 9 and 10, carbons 9 and 11, carbons 8 and 14, or carbons 14 and 15 indicates a double bond, with the proviso that there be no adjacent double bonds on a ring or adjacent rings (e.g., if a double bond is present between carbons 8 and 9, no other double bonds are present in either of the two adjacent rings, or double bonds are not co-present between carbons 8 and 14 and carbons 14 and 15), and/or R³ is H if a double bond is present between carbons 9 and 10 and/or R⁷ is H if a double bond is present between carbons 8 and 14 or carbons 14 and 15; and formula II having a formula as:

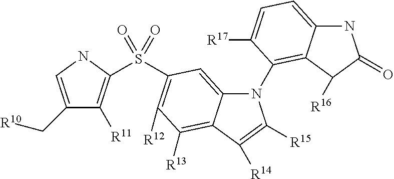

a prodrug or pharmaceutically acceptable salt thereof.

For example, the compound of the inventive method or composition is a compound of formula IA:

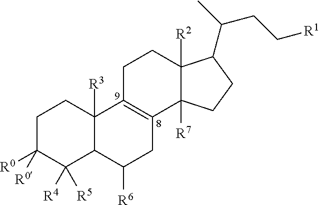

wherein:
R0 and R0′ is hydroxyl, —OSO3H, —OSO3-, —OCOCH3, —OPO3H, —OPO3-, or hydrogen, or R0 and R0′ together represent a carbonyl group;

R¹ is a linear or branched alkyl, aryl, alkene, alkyne, a substituted alkene, a. substituted alkyl, a substituted alkyne, a substituted aryl, an alkyl halide, alkoxy such as an alcohol or an aryloxy, or an acetyl or ester group having from 2 to 6 carbon;
R², R³, R⁴, R⁵, R⁷ are each H or Me;
R⁶ is H or Me or OH or oxo (═O) or halide;
a prodrug or pharmaceutically acceptable salt thereof.

In various embodiments, the compound is a cholesterol precursor. In other embodiments, the compound is a cholesterol intermediate in the cholesterol biosynthesis. [The prodrug refers carrier moiety covalently bound to a compound comprising structural Formula I, II, III, IV, or other structures of the compounds of formula composites. The active compound can be released from the carrier portion of the composite under in vitro or in vivo conditions. Compounds known in the art form prodrugs may be, for example, in the following article example: Sloan, K B, Prodrugs, M. Dekker, New York, 1992; and Testa, B. and Mayer, J M, Hydrolysis in drug and prodrug metabolism: chemistry, biochemistry, and enzymology, Wiley-VCH, Zurich, 2003.

In various embodiments, the compound is a cholesterol intermediate in the cholesterol biosynthesis. Alternatively, the compound is a compound listed below:

Alternatively, the compound is a compound listed in FIGS. 19a and 19b.

Any prodrug or pharmaceutically acceptable salt of the above compounds are contemplated to be within the scope of the invention.

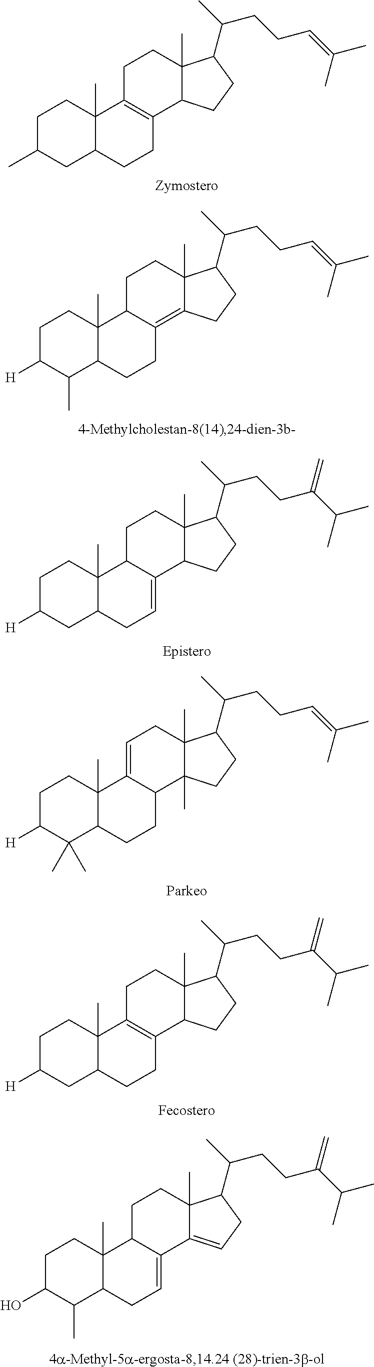

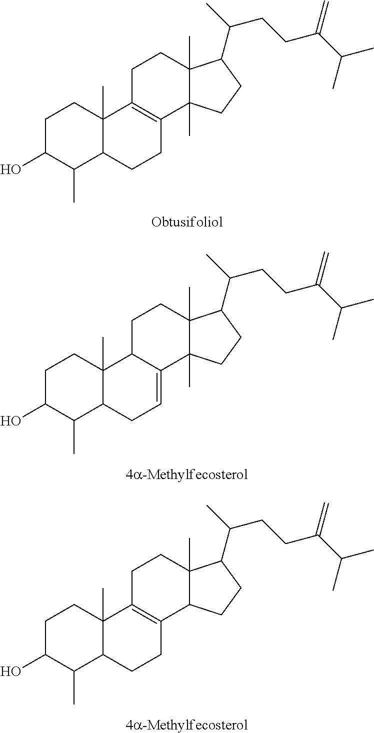 [SMILES: C=C(CCC(C)C1CCC2(C)C3=C(CCC12C)C1(C)CCC(O)C(C)C1CC3)C(C)C ; C=C(CCC(C)C1CCC2(C)C3=CCC4C(C)C(O)CCC4(C)C3CCC12C)C(C)C ; C=C(CCC(C)C1CCC2C3=C(CCC21C)C1(C)CCC(O)C(C)C1CC3)C(C)C]

### Pharmaceutical Compositions

In some embodiments of the invention, pharmaceutical compositions of one or more therapeutic compounds can be prepared by formulating one or more of these therapeutic compounds in a pharmaceutically acceptable carrier. As used herein, “pharmaceutically or therapeutically acceptable carrier” refers to a carrier medium which does not interfere with the effectiveness of the biological activity of the active ingredients and which is not toxic to the host or patient. The type of carrier which is used in the pharmaceutical preparation will depend on the method by which the therapeutic compounds are to be administered. Many methods of preparing pharmaceutical compositions for various routes of administration are well known in the art.

As used herein, “pharmaceutically acceptable ophthalmic carrier” refers to a pharmaceutically acceptable excipient, carrier, binder, and/or diluent for delivery of the compound of the structural formula 1 directly or indirectly to, on or near the eye. Accordingly, the invention further comprises a composition comprising the compound of the structural formula I or formula II and a pharmaceutically acceptable ophthalmic carrier.

Optionally, the composition includes a free acid, free base, salt (e.g., an acid or base addition salt), hydrate or prodrug of the compound of structural formula I or formula II. The phrase “pharmaceutically acceptable salt” or “pharmaceutically acceptable acid,” as used herein, refers to pharmaceutically acceptable organic or inorganic salts or acids, respectively, of a compound of formula I or formula II. The counter ion may be any organic or inorganic moiety that stabilizes the charge on the parent compound. Furthermore, a pharmaceutically acceptable salt (or acid) may have more than one charged atom in its structure. Instances where multiple charged atoms are part of the pharmaceutically acceptable salt (or acid) can have multiple counter ions. Hence, a pharmaceutically acceptable salt (acid) can have one or more charged atoms and/or one or more counter ion.

Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion.

The prodrug is a material that includes the compound of structural formula I or formula II covalently bound to a carrier moiety. The carrier moiety can be released from the compound of structural formula 1, in vitro or in vivo to yield compound of structural formula I or formula II. Prodrug forms are well known in the art as exemplified in Sloan, K. B., Prodrugs, M. Dekker, New York, 1992; and Testa, B. and Mayer, J. M., Hydrolysis in drug and prodrug metabolism: chemistry, biochemistry, and enzymology, Wiley-VCH, Zurich, 2003.

In some embodiments of the invention, pharmaceutical compositions are prepared by dissolving the invention composition in an appropriate solvent. Appropriate solvents include, but are not limited to, water, saline solution (for example, NaCl), buffered solutions, ointments, gels or other solvents. In certain embodiments, the solvents are sterile.

Aqueous solutions and diluents for suspensions that are used in preparation of eye drops can include distilled water, physiological saline, and the like. These pharmaceutical compositions can be formulated by admixing, diluting or dissolving the compound, optionally, with appropriate pharmaceutical additives such as excipients, disintegrators, binders, lubricants, diluents, buffers, antiseptics, moistening agents, emulsifiers, dispersing agents, stabilizing agents and dissolving aids in accordance with conventional methods and formulating in a conventional manner depending upon the dosage form. Non-aqueous solutions and diluents for suspensions can include edible (eg vegetable) oil, liquid paraffin, mineral oil, propylene glycol, p-octyldodecanol, polysorbate, macrogols, aluminum monostearate as well as similar solvents.

Various additives may be contained in eye drops, ophthalmic gels and/or ophthalmic ointments as needed. These can include additional ingredients, additives or carrier suitable for use in contact on or around the eye without undue toxicity, incompatibility, instability, irritation, allergic response, and the like. Additives such as solvents, bases, solution adjuvants, suspending agents, thickening agents, emulsifying agents, stabilizing agents, buffering agents, isotonicity adjusting agents, pH-adjusting agents, chelating agents, soothing agents, preservatives, corrigents, flavoring agents, coloring agents, excipients, binding agents, lubricants, surfactants, absorption-promoting agents, dispersing agents, preservatives, solubilizing agents, and the like, can be added to a formulation where appropriate.

For example, eye drops can be formulated by dissolving the compound in sterilized water in which a surface active agent is dissolved and optionally adding appropriate pharmaceutical additives such as a preservative, a stabilizing agent, a buffer, an antioxidant and a viscosity improver.

For example, buffering agents are added to keep the pH constant and can include pharmaceutically acceptable buffering agents such as borate buffer, citrate buffer, tartrate buffer, phosphate buffer, acetate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution. Buffering agents are included in an amount that provides sufficient buffer capacity for the expected physiological conditions.

Other buffers include, but are not limited to, buffers based on HEPES (N-{2-hydroxyethyl}peperazine-N′-{2-ethanesulfonic acid}) having pKa of 7.5 at 25° C. and pH in the range of about 6.8-8.2; BES (N,N-bis{2-hydroxyethyl}2-aminoethanesulfonic acid) having pKa of 7.1 at 25° C. and pH in the range of about 6.4-7.8; MOPS (3-{N-morpholino}propanesulfonic acid) having pKa of 7.2 at 25° C. and pH in the range of about 6.5-7.9; TES (N-tris{hydroxymethyl}-methyl-2-aminoethanesulfonic acid) having pKa of 7.4 at 25° C. and pH in the range of about 6.8-8.2; MOBS (4-{N-morpholino}butanesulfonic acid) having pKa of 7.6 at 25° C. and pH in the range of about 6.9-8.3; DIPSO (3-(N,N-bis{2-hydroxyethyl}amino)-2-hydroxypropane)) having pKa of 7.52 at 25° C. and pH in the range of about 7-8.2; TAPS ({(2-hydroxy-3{tris(hydroxymethyl)methylamino}-1-propanesulfonic acid)) having pKa of 7.61 at 25° C. and pH in the range of about 7-8.2; TAPS ({(2-hydroxy-1,1-bis(hydroxymethyl)ethyl)amino}-1-propanesulfonic acid)) having pKa of 8.4 at 25° C. and pH in the range of about 7.7-9.1; TABS (N-tris(hydroxymethyl)methyl-4-aminobutanesulfonic acid) having pKa of 8.9 at 25° C. and pH in the range of about 8.2-9.6; AMPSO (N-(1,1-dimethyl-2-hydroxyethyl)-3-amino-2-hydroxypropanesulfonic acid)) having pKa of 9.0 at 25° C. and pH in the range of about 8.3-9.7; CHES (2-cyclohexylamino)ethanesulfonic acid) having pKa of 9.5 at 25° C. and pH in the range of about 8.6-10.0; CAPSO (3-(cyclohexylamino)-2-hydroxy-1-propanesulfonic acid) having pKa of 9.6 at 25° C. and pH in the range of about 8.9-10.3; and CAPS (3-(cyclohexylamino)-1-propane sulfonic acid) having pKa of 10.4 at 25° C. and pH in the range of about 9.7-11.1.

In addition to a buffer, isotonizers can be added to eye drops to make the preparation isotonic with the tear. Isotonizers include, but are not limited to, sugars such as dextrose, glucose, sucrose and fructose; sugar alcohols such as mannitol and sorbitol; polyhydric alcohols such as glycerol, polyethylene glycol and propylene glycol; and salts such as sodium chloride, sodium citrate, benzalkonium chloride, phedrine chloride, potassium chloride, procaine chloride, chloram phenicol, and sodium succinate. Isotonizers are added in an amount that makes the osmotic pressure of the eye drop equal to that of the tear.

Preservatives can be added to maintain the integrity of the eye drop and/or ophthalmic ointment. Examples of preservatives include, but are not limited to, sorbic acid, benzalkonium chloride, benzododecinium bromide, parabens, chlorobutanol, benzylic alcohol, phenylethyl alcohol, edentate disodium, sorbic acid, polyquaternium-1, or other agents known to those skilled in the art.

In some embodiments, thickeners are used to increase the viscosity of ophthalmic preparations such as eye drops, ophthalmic gels and/or ophthalmic ointments. Thickeners that can be used include, but are not limited to, glycerol, polyethylene glycol, carboxymethyl cellulose and carboxyvinyl polymers.

In addition to the above, in some embodiments, it is desirable to use additional agents which include, but are not limited to, stabilizers such as sodium sulfite, sodium carbonate, and propylene glycol; antioxidants such as ascorbic acid, sodium ascorbate, butylated hydroxy toluene (BHT), butylated hydroxyanisole (BHA), tocopherol, sodium thiosulfate; and/or chelating agents such as ethylene-diamine-tetra-acetic acid (EDTA), ethylene glycol-bis-(2-aminoethyl)-N,N,N,N-tetraacetic acid (EGTA) and sodium citrate.

Eye drops, ophthalmic gels and/or ophthalmic ointments can be prepared by aseptic manipulation or alternatively sterilization is performed at a suitable stage of preparation. For example, a sterile pharmaceutical composition can be prepared by mixing sterile ingredients aseptically. Alternatively, the sterile pharmaceutical composition can be prepared by first mixing the ingredients then sterilizing the final preparation. Sterilization methods can include, but are not limited to, heat sterilization, irradiation and filtration.

Ophthalmic ointments (eye ointments) can be aseptically prepared by mixing the active ingredient into a base that is used for preparation of eye ointments followed by formulation into pharmaceutical preparations with any method known in the art. Typical bases for eye ointments are exemplified by vaseline, jelene 50, plastibase and macrogol. In addition, surfactants may be added to increase hydrophilia.

A number of effective methods for controlled release of an active agent are available. See, for example, Wagh V. D., Inamdar B., Samanta M. K., Polymers used in ocular dosage form and drug delivery systems. Asian J Pharm 2, 2008, 12-17 and the literature references cited therein, the contents of which are incorporated herein by reference. The use of polymers (e.g., cellulose derivatives such as hydroxypropylmethylcellulose (HPMC) and hydroxypropylcellulose (HPC), poly(acrylic acid) (PAA), polyacrylates, cyclodextrins and natural gums, polyorthoesters (POEs) and mucoadhesive polymers); semisolids such as gels, films and other inserts; resins such as ion exchange resins; iontophoretic delivery; and colloidal particles such as microspheres and nanoparticles, are specifically contemplated.

The compounds of the invention may also be provided in combination with other therapeutic agents. In some embodiments, the compounds of the invention may be co-formulated with other active agents, including, but not limiting to, anti-infective agents, antibiotics, antiviral agents, anti-fungal, anti-protozoal agent, anti-inflammatory drugs, anti-allergic agents including anti-histamines, artificial tears vasoconstrictors, vasodilators, local anesthetics, analgesics, intraocular pressure-lowering agents, immunoregulators, anti-oxidants, vitamins and minerals, an enzyme inhibitor or alternatively, proteases and peptidases, a cytokine inhibitor, and the like.

In various embodiments, the compounds of the invention may also be provided in combination with an ocular therapeutic selected from the group consisting of Acular (ketorolac tromethamine ophthalmic solution) 0.5%, Acuvail (ketorolac tromethamine), AK-Con-A (naphazoline ophthalmic), Akten (lidocaine hydrochloride), Alamast, Alphagan (brimonidine), Alrex, Astepro (azelastine hydrochloride nasal spray), AzaSite (azithromycin), Bepreve (bepotastine besilate ophthalmic solution), Besivance (besifloxacin ophthalmic suspension), Betaxon, BSS Sterile Irrigating Solution, Cosopt, Durezol (difluprednate), Eylea (aflibercept), Lotemax, Lucentis (ranibizumab), Lumigan (bimatoprost ophthalmic solution), Macugen (pegaptanib), Ocuflox (ofloxacin opthalmic solution) 0.3%, OcuHist, Ozurdex (dexamethasone), Quixin (levofloxacin), Rescula (unoprostone isopropyl ophthalmic solution) 0.15%, Restasis (cyclosporine ophthalmic emulsion), Salagen Tablets, Travatan (travoprost ophthalmic solution), Valcyte (valganciclovir HCl), Viroptic, Vistide (cidofovir), Visudyne (verteporfin for injection), Vitrasert Implant, Vitravene Injection, ZADITOR, Zioptan (tafluprost ophthalmic solution), Zirgan (ganciclovir ophthalmic gel), Zymaxid (gatifloxacin ophthalmic solution), Atropine, Flurbiprofen, Physostimine, Azopt, Gentamicin, Pilocarpine, Bacitracin, Goniosol, Polymyxin B, Betadine, Gramicidin, Prednisolone, Betaxolol, Humorsol, Proparacaine, Betoptic, Hylartin, Propine, Brinzolamide, Hypertonic NaCl, Puralube, BSS, Indocycanine Green, Rose Bengal, Carbachol, Itraconazole, Sodium Hyaluronate, Cefazolin, Latanoprost, Suprofen, Celluvisc, Mannitol, Terramycin, Chloramphenicol, Methazolamide, Timolol, Ciloxan, Miconazole, Tobramycin, Ciprofloxacin, Miostat, Triamcinolone, Cosopt, Muro 128, Trifluridine, Demecarium, Neomycin, Tropicamide, Dexamethasone, Neptazane, Trusopt, Dipivefrin, Ocuflox, Vidarabine, Dorzolamide, Ofloxacin, Vira-A, Epinephrine, Oxytetracycline, Viroptic, Fluorescein, Phenylephrine, and Xalatan.

### Kits

Some embodiments of the invention relate to kits for preventing and/or ameliorating one or more symptoms associated with an eye disease. The kits can comprise one or more containers that contain one or more of the therapeutic compounds described herein. The compounds can be present in the container as a prepared pharmaceutical composition, or alternatively, the compounds can be unformulated. In such embodiments, the kit can include the unformulated compounds in a container that is separate from the pharmaceutically acceptable carrier. Prior to use, the compound in diluted or otherwise mixed with the pharmaceutically acceptable carrier.

Some embodiments of the kits provided herein also comprise instructions which describe the method for administering the pharmaceutical composition in such a way that one or more symptoms associated with an eye disease which includes, but is not limited to, retinal degeneration, presbyopia, cataracts and/or nuclear sclerosis of the eye lens. In some embodiments, the instructions also describe the procedure for mixing the therapeutic compounds contained in the kit with ophthalmic pharmaceutically acceptable carriers.

In some embodiments of the invention, the container that comprises the therapeutic compounds described herein is a container which is used for ophthalmic administration. In certain embodiments, the container is a dropper for administering eye drops. In other embodiments, the container is a tube for administering an ophthalmic gel or an ophthalmic ointment.

Some embodiments of this invention are further illustrated by the following examples that should not be construed as limiting. It will be appreciated by those of skill in the art that the techniques disclosed in the examples which follow represent techniques discovered by the inventor to function well in the practice of the embodiments of the invention described herein, and thus can be considered to constitute preferred modes for the practice of these embodiments. Those of skill in the art will, however, in light of the present disclosure, appreciate that many changes can be made in the specific embodiments which are disclosed herein and still obtain a like or similar result without departing from the spirit and scope of the invention.

### Devices

Some embodiments of the invention relate to devices for administering the invention sterol to a subject. In some embodiments, the devices include and interior portion, cavity or reservoir that contains the invention sterol formulated in a pharmaceutically acceptable carrier. In such embodiments, the pharmaceutically carriers include, but are not limited to, solutions, gels and ointments. In certain embodiments, the interior portion, cavity or reservoir contains one or more of the invention sterol -containing pharmaceutical preparations described herein.

In some embodiments, the devices contemplated herein also comprise an applicator that is coupled to the interior portion, cavity or reservoir of the device. The applicator can be cylindrical, conical or any other shape that permits the invention sterol-containing pharmaceutical preparation to be delivered from the interior portion, cavity or reservoir to the eye. In a preferred embodiment, the applicator is a tapered cylinder wherein the wide end is coupled to the interior portion, cavity or reservoir and the tapered end forms the exit opening for passage of the invention sterol-containing pharmaceutical preparation to the eye.

In yet an alternate embodiment, the invention provides a method (and compositions thereof) of treating or preventing vision disorders, the method comprising administering to an individual in need thereof an effective amount of a composition comprising a compound of formula III:

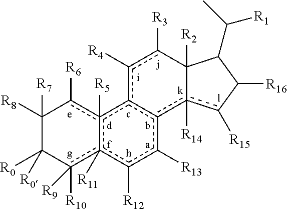

prodrug or pharmaceutically acceptable salt thereof,
wherein:
R0 and R0′ is hydroxyl, —OSO₃H, —OSO₃⁻, —OCOCH₃, —OPO₃H, —OPO-₃⁻, or hydrogen, or R0 and R0′ together represent a carbonyl group;
R1 is alkyl, aryl, substituted aryl, alkenes, alkynes, substituted olefins, substituted alkyl, substituted alkyne, alkyl halide, alkoxy such as an alcohol, or aryloxy, acetyl group, ester group, biphenyl, substituted biphenyl, benzyl, substituted benzyl, benzoyl, or substituted benzoyl group;
R2, R5, R11 and R14 is H or Me, when they are not connected to a carbon which forms a double bond;
R9 and R10 is H, alkyl, substituted alkyl, or hydroxyl, or R9 and R10 together represent a carbonyl, when R9 and R10 are not connected to carbons which form a double bond;
R9 and R10 is H, alkyl, substituted alkyl, when R9 and R10 are connected to carbons which form a double bond;
R12 is H, an alkyl group, a hydroxyl group, an acyl group, or a substituted alkyl group;
R3, R4, R6, R7, R8, R13, R15, R16 is H, alkyl, or a haloalkyl group;
wherein, the dashed line portion, in between a, b, c, d, e, f, g, h, i, j, k, 1 of the adjacent carbon atoms containing one, two or three double bonds.

The invention provides a method of treating or preventing vision disorders, the method comprising administering to an individual in need thereof an effective amount of a composition comprising a compound of formula IV:

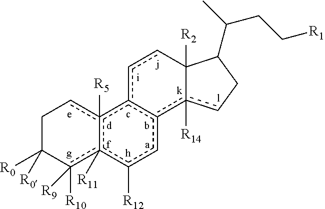

prodrug or pharmaceutically acceptable salt thereof,
wherein:
R0 and R0′ is hydroxyl, —OSO₃H, —OSO₃⁻, —OCOCH₃, —OPO₃H, —OPO₃⁻, or hydrogen, or R0 and R0′ together represent a carbonyl group;
R1 is alkyl, aryl, substituted aryl, alkenes, alkynes, substituted olefins, substituted alkyl, substituted alkyne, alkyl halide, alkoxy such as an alcohol, or aryloxy, acetyl group, ester group, biphenyl, substituted biphenyl, benzyl, substituted benzyl, benzoyl, or substituted benzoyl group; R2, R5, R11 and R14 is H or Me, when they are not connected to a carbon which forms a double bond;
R9 and R10 is H, alkyl, substituted alkyl, or hydroxyl, or R9 and R10 together represent a carbonyl, when R9 and R10 are not connected to carbons which form a double bond;
R9 and R10 is H, alkyl, substituted alkyl, when R9 and R10 are connected to carbons which form a double bond;
R12 is H, an alkyl group, a hydroxyl group, an acyl group, or a substituted alkyl group;
wherein, the dashed line portion, in between a, b, c, d, e, f, g, h, i, j, k, l of the adjacent carbon atoms containing one, two or three double bonds.

The invention provides a method of treating or preventing vision disorders, the method comprising administering to an individual in need thereof an effective amount of a composition comprising a compound of formula I:

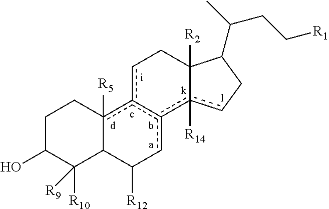

a prodrug or pharmaceutically acceptable salt thereof.
wherein:
R1 is alkyl, aryl, substituted aryl, alkenes, alkynes, substituted olefins, substituted alkyl, substituted alkyne, alkyl halide, alkoxy such as an alcohol, or aryloxy, acetyl group, ester group, biphenyl, substituted biphenyl, benzyl, substituted benzyl, benzoyl, or substituted benzoyl group; R2, R5, and R14 is H or Me, when they are not connected to a carbon which forms a double bond;
R9 and R10 is H, alkyl, substituted alkyl, or hydroxyl, or R9 and R10 together represent a carbonyl;
R12 is H, an alkyl group, a hydroxyl group, an acyl group, or a substituted alkyl group; wherein, the dashed line portion, in between a, b, c, d, i, k, l of the adjacent carbon atoms containing one, two or three double bonds.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are intended to illustrate but not to limit the invention in any manner, shape, or form, either explicitly or implicitly. While they are typical of those mat might be used, other procedures, methodologies, or techniques known to those skilled in the art may alternatively be used.

### Methods

The effect of lanosterol or cholesterol on the aggresome formation of various crystallins was studied by transfecting the cells using plasmids containing various crystallin genes. The cells were cultivated for 24 h to enable efficient protein expression and aggresome formation. Then the cells were treated with 0-40 μM sterols in 2% DMSO. The cells treated with 2% DMSO were used as the control. After treatment for 2 h, the cells were moved to fresh DMEM medium containing 10% fetal bovine serum and further cultivated for 12 h. Then the cells were used for microscopy analysis.

The microscopy samples were prepared by washing the slips by phosphate buffered saline (PBS) three times. The cells were fixed with 4% paraformaldehyde for 40 min followed by three times washing with PBS. The cells were permeabilized with 0.1% Triton X-100 (Sigma) in PBS for 10 min and blocked with 5% normal goat serum in PBS for 1 h at 37° C. Immunostaining was carried out by adding mouse anti-Flag antibody (1:500) or anti-p62 antibody (1:200) in PBS buffer containing 5% normal goat serum and cultivated for 1 h at 37° C. Then the slips were washed three times with PBS, and further incubated with Alexa 649-conjugated goat anti-mouse IgG (1:250) for 1 h at ambient temperature. The nuclei were counterstained with Hoechst 33342. The mounted cells were analyzed using a Carl Zeiss LSM 710 confocal microscope.

### Lipid Extraction of the Cells

Extraction of lipids was performed using Bligh and Dyer method 17. In brief, 1×106˜107 HeLa cells were washed 3-5 times with PBS and then scraped in 400 μl ice-cold methanol and transferred to a 1.5 ml Eppendorf tube with the addition of 200 μl chloroform. The samples were vortex-agitated for 1 min and then mixed with 300 μl of 1M KCL. The organic and aqueous phases were separated by microcentrifugation at 14 000 r.p.m. for 5 min at 4° C. After separation, the lower organic phase was collected. Then the residual aqueous phase was re-extracted twice using 300 μl chloroform. The collected organic phases were dried using a SpeedVac sample concentrator under vacuum. The dried samples were stored at −80° C. for further LC/MS analysis.

### LC/MS Analysis

The dried lipid extracts were re-suspended in 100 μl methanol. The samples were vortex-agitated for 10 min, treated by 80 W ultrasonic for 30 min, microcentrifuged at 14000 r.p.m. for 10 min, and then the supernatant was transferred to a new Eppendorf tube. The microcentrifugation treatment was repeated for three times. The derivatized samples were analyzed by an Agilent 1290/6460 triple quadrupole LC/MS using an alternative Atmospheric Pressure Chemical Ionisation (APCI) source. The lipids were separated using an Agilent SB-C18 column. Selective ion monitoring was performed using the electron ionization mode. The highly pure lanosterol and cholesterol were used as the controls. The MS determination was performed using a gas temperature of 350° C., a gas flow rate of 4 L/min, a nebulizer of 60 psi, a vaporizer of 350° C., a capillary of 3500 V and a coroua current of 4 μA. To optimize the sensitivity and specificity, two qualifier ions were selected for the MS analysis of each compound (369.3/161.1 and 369.3/147 for cholesterol, and 409.2/191.3 and 409.2/109 for lanosterol).

### Western Blotting

The cell lysates were prepared in RIPA buffer containing 50 mM Tris (pH 8.0), 150 mM NaCl, 1% Triton X-100, 1 mM EDTA, 0.5% sodium deoxycholate and 0.1% SDS. The supernatant and precipitation fractions were separated by centrifugation. The proteins were separated by a 12.5% SDS-PAGE and transferred to a PVDF membrane (GE). The antibodies against Flag or GFP were used to identify the overexpressed LSS and crystallin proteins, respectively. Quantification of the Western blot bands was achieved by the software GELPRO. The presented quantitative data were calculated from three independent experiments.

### Protein Expression and Purification

The recombinant his-tagged WT and mutated β- and γ-crystallin proteins were overexpressed in E. coli Rosetta and purified by Ni-NTA affinity column followed by gel filtration chromatography using the same protocol as those described elsewhere 13,14,16,18. The overexpression and purification of the non-tagged αA- and αB-crystallins were performed as described previously 19. The purity of the proteins were estimated to be above 95% as evaluated by one homogeneous band on 12.5% SDS-PAGE, 10% native-PAGE and a single peak in the size-exclusion chromatographyprofile. The protein concentration was determined according to the Bradford method by using BSA as the standard 20. All protein samples were prepared in 20 mM PBS buffer containing 150 mM NaCl, 1 mM EDTA and 1 mM DTT. Protein aggregation and aggregates dissociation

The aggregates of the WT and mutated αA- and αB-crystallin proteins were obtained by heating the proteins solutions containing 1 M guanidine chloride (ultrapure, Sigma) with a concentration of 5 mg/ml at 60° C. for 48 h. The aggregates of the WT and mutated α- and γ-crystallins were prepared by heating the protein solutions at pH 3 and 37° C. for 48 h. The formation of aggregates was confirmed by turbidity (absorbance at 400 nm) and transmission electron microscope (EM) measurements. The preformed aggregates were resuspended in 20 mM PBS with a final concentration of 0.1 mg/ml (approximately 5 μM). The resuspended aggregates were treated by 50 μM lanosterol or cholesterol in liposomes formed by 50 μM DPPS at 37 ° C. After 72 h treatment, the protein solutions were used for negative-stained EM observations. After 144 h incubation, the protein solutions were used for turbidity measurements and determinations of protein concentrations in the soluble fractions. The soluble proteins were determined by centrifuge the protein solutions to separate the supernatant and precipitation fractions. Then the protein concentration in the supernatant was determined by the Bradford method. The EM samples were prepared by depositing the protein solutions onto a freshly glow-discharged carbon coated copper grid. Negative-staining samples were obtained by staining the grid with 1.25% uranyl acetate for 30 s. The negatively stained EM pictures were obtained on a Hitachi H-7650B transmission electron microscope with a voltage of 120 kV and a magnification of 68000.

### Animal Study

This study was approved by IACUC. Rat eyeballs were fixed with 4% paraformaldehyde in PBS overnight at 4° C. The samples were cryoprotected with 30% sucrose in PBS and embedded in OCT compound (Sakura Finetechnical, Tokyo, Japan). These tissues were sliced with a Microm HM 560 cryostat microtome (Microm Laborgeräte GmbH, Walldorf, Germany) into 14 μm sections and then incubated with 0.1% Toluidine blue in PBS. After washing in PBS three times, the sections were mounted with Glycerol/PBS (1:1). Sections were viewed and images were captured using a Zeiss Observer Al microscope.

### Treatment of Rabbit Cataract Lens

Rabbits were euthanized by CO2 inhalation and lenses were immediately dissected and were treated with vehicle or lanosterol dissolved in vehicle to make 5 mM solutions. Lens tissues were incubated in these solutions for 6 days in the dark at room temperature. Cataract were examined under a microscope and then photographed. Degree of cataract was assessed using AREDS cataract grading system.

### Treatment of Dog Cataract Lens

Grading System of Dog Cataract
Grade 0: absence of opacification (no cataract);
Grade 1: a slight degree of opacification (incipient stage);
Grade 2: presence of diffuse opacification involving almost the entire lens (immature stage);
Grade 3: presence of extensive thick opacification involving the entire lens (mature stage)

To assess the effect of lanosterol treatment on cataract in live animals, left eye of dogs with bilateral cataract were treated for topical eye drops. The right eye of each animal was administered one drop of vehicle, 3 times a day so that the drop coated the eye. One drop of lanosterol was administered to the left eye of each animal in the same manner. Both eyes of some control animals were treated for vehicle. The drops were administered in 3 times in a 50-μl drop/each application per day over 6 weeks. Cataract were examined by slit lamp and photographed. Prior to observation, pupils were dilated with tropicamide. Each lens was recorded by photography. Degree of cataract was assessed using AREDS cataract grading system.

### Treatment of Cataract Lenses in Dogs

This study was approved by IACUC of Zhongshan Ophthalmic Center and West China Hospital. The following adult dog breeds were used for assessing the treatment effect: Black Labrador, Queensland Heeler, Miniature Pincher. All dogs were non-diabetic and had normal ocular surfaces and ocular adnexa, with naturally occurring adult onset cataract. We screened all exons of the LSS gene in these dogs and we did not find any mutations. To assess the effect of lanosterol treatment on cataract in live animals, dogs were pre-medicated with intramuscular injection of acepromaxine and butorphanol. After 20 mins, induction of anesthesia was performed by IV propofol. Dogs were then immediately intubated and maintained on oxygen and 2% isoflurane at 2 liters per minute. Lanosterol (100 μg) loaded nanoparticles were injected into the vitreous cavity in the test eye using a 28-gauge needle. The control eye was given an injection with empty nanoparticle carriers as a negative control. The treatment eyes were treated with lanosterol in topical eye drops (see below for eye drop formulation). One drop of lanosterol was administered three times daily to the test eye in a 50-μl per drop/each application over 6 weeks. weeks. Degree of cataract was examined by slit lamp and photographed at the beginning and the end of 6-week treatment period. Prior to examinations, pupils were dilated with 1% Tropicamide and 10% Phenylephrine. Degree of cataract was assessed by a blinded examiner and scored based on canine cataract stage, shown below. Improvements in lens clarity and transparency were quantified.

### Preparation of Drug-Loaded Nanoparticles

Lanosterol was loaded into a lipid-polymer hybrid nanoparticle through an established nanoprecipitation method. Specifically, lanosterol at the desired concentration was added to a 2.5 mg/mL polycaprolactone (PCL) solution in acetonitrile. Lecithin and 1,2-distearoyl-snglycero-3-phosphoethanolamine-Ncarboxy(polyethylene glycol) 2000 (DSPE-PEG-COOH) (molar ratio=7.5:2.5) were dissolved in 4% ethanol aqueous solution at 20% of the PCL polymer weight and heated to 65° C. The lanosterol/PCL solution was then added into the preheated lipid solution drop-wise under gentle stirring then rigorous vortexing for 3 min.

The mixture solution was then stirred for 2 h to allow the nanoparticles to form and the acetonitrile to evaporate. Next, the nanoparticle solution was washed three times using an Amicon Ultra-4 centrifugal filter (Millipore, Billerica, Mass.) with a molecular weight cut-off of 10 kDa to remove the remaining organic solvent and free molecules. The resulting nanoparticles were then re-suspended in PBS buffer for subsequent uses. The size, size distribution, and surface zeta potential of the drug-loaded nanoparticles were characterized by dynamic light scattering. The loading yield of lanosterol was quantified by high performance liquid chromatography.

| | | |
|---|---|---|
| Hydroxypropyl-β-Cyclodextrin | 165 | g |
| Polysorbate 80 | 1 | g |
| EDTA2Na | 1.1 | g |
| Alkyldimethylbenzylammonium chloride | 0.055 | g |
| EtOH | 200 | ml |

Then add ddH₂O till the final volume is 1.1L (PH 5.66)

| | | |
|---|---|---|
| Lanosterol | 2.5 | g |
| Hydroxypropyl-β-Cyclodextrin | 165 | g |
| Polysorbate 80 | 1 | g |
| EDTA2Na | 1.1 | g |
| Alkyldimethylbenzylammonium chloride | 0.055 | g |
| EtOH | 200 | ml |

Then add ddH2O till the final volume is 1.1L (PH 5.66)
25mM lanosterol excipient solution formulation:

| | | |
|---|---|---|
| Lanosterol | 12.5 | g, |
| hydroxypropyl-β-cyclodextrin | 165 | g |
| polyethylene polysorbate 80 | 1 | g |
| EDTA2Na | 1.1 | g |
| benzalkonium chloride | 0.055 | g |
| ethanol | 200 | ml |

followed by addition of double distilled water until a final volume of 1.1L was reached (PH value of 5.66).

### Alternate Representative Formulations for Lanosterol

Tetracyclic triterpenoid can be prepared from a chain of squalene cyclization. Many tetracyclic triterpenoids are cholesterol biosynthetic intermediates, soluble in chloroform, ethanol, ethyl ether. The project team evaluated previous medicinal records of lanosterol eye drops, and determined its optimal concentration. Based on the physical and chemical properties of lanosterol, we screened many possible prescription eye drops formulations and improved the production process, and designed the four prescriptions for the subsequent pharmacology and pharmaceutical research at the same time. According to CFDA and ICH requirements, lanosterol eye drops pharmaceutical research programs were conducted as follows:
1. Lanosterol Eye Drops Formulation

Lanosterol was dissolved in small amount of ethanol, mixed well and added to PBS to prepare lanosterol formulations in buffered saline system. The formulations were then screened using orthogonal design. The optimal formulation was selected based on the stability of the active ingredient, irritation, and intraocular bioavailability of the eye drops. The active ingredient concentration and the excipients were finalized, with major excipients including: potassium biphosphate, disodium hydrogen phosphate, sodium chloride and potassium chloride, benzalkonium chloride.
2: Lanosterol Vehicle Formulation Eye Drops

Lanosterol was dissolved in 2% of Transtol HP, with polysorbate 80 and hydrogenated castor oil being added as a solubilizer and suspending agents. Water for injection (WFI) was gradually added until a clear solution was prepared. The optimal formulation was selected based on the stability of the active ingredient, irritation, and intraocular bioavailability of the eye drops. The active ingredient concentration and the excipients were finalized, with major excipients including: polysorbate 80, hydrogenated castor oil, polyethylene glycol, Thai Shamrock Park, sodium citrate, benzalkonium chloride. p0 3: Sustained-Release Lanosterol Eye Drops

A mucoadhesive polymer drug delivery system was developed for lanosterol, using Salvia and polycarbophil. The newly developed system lead to improved solubility and stability, compared to lanosterol in pure aqueous solution. Meanwhile, Salvia as a traditional Chinese medicine, exhibited many benefits such as soothing the nerves and heart, and analgesic effect. When it was used to prepare Lanosterol ophthalmic formulations, Salvia helps enhance lanosterol immunosuppressive effects and eye tissues penetration. In addition, the new system maintained effective therapeutic levels of the active drug for prolonged ocular residence time, therefore significantly reduced the frequency of administration, effectively simplified dosing regimen, and increased patient compliance and treatment success rates. The optimal formulation was selected based on the stability of the active ingredient, irritation, and intraocular bioavailability of the eye drops. The active ingredient concentration and the excipients were finalized, with major excipients including: Salvia, polycarbophil, polysorbate 80, polyvinyl alcohol, sodium chloride, mannitol, sodium citrate, benzalkonium chloride.
4: Lanosterol Emulsion Formulation

After being suspended in castor oil, lanosterol was pulverized into nanoparticles, using a high pressure homogenizer. A stable emulsions was prepared by adding polysorbate 80 and glycerol as solubilizer and suspending agents. The emulsion overcome the challenge of poor stability and low solubility of lanosterol as it was dissolved in pure aqueous solution. The optimal formulation was selected based on the stability of the active ingredient, irritation, and intraocular bioavailability of the eye drops. The active ingredient concentration and the excipients were finalized, with major excipients including: castor oil, polysorbate 80, glycerin, polyethylene glycol, Thai Shamrock Park, edetate disodium, sodium chloride, benzalkonium chloride.

Sterol Ligands

The AutoDockTools package was used to generate input files for the computational docking runs and ligand site characterization. Docking was conducted using Autodock 4.0 against both the human alphaB crystallin crystal structure (protein databank code: 2WJ7) and the solid-state NMR structure of the alpha-crystallin domain in alphaB-crystallin oligomers (protein databank code: 2KLR), using a search space enclosing the entire protein structure in a 1.0 Å grid, and Lamarckian genetic algorithm starting with an initial population of 500 randomly positioned inputs of the small molecule compound being docked. The maximum number of energy evaluations was set to 2.5 107 and used a mutation rate of 0.02 with a crossover rate of 0.8, and results were clustered at 2.0-Å root mean square deviation.
Human AlphaB Crystallin crystal structure docking calculation:
Lanosterol −10.3 Kcal/mol
Lanthosterol −10.4 Kcal/mol
Ergsterol −10.4 Kcal/mol
Zymosterol −10.4 Kcal/mol
Parkeol −10.6 Kcal/mol
Solid-state NMR structure of the alpha-crystallin domain
Lanosterol −6.9 Kcal/mol
Lanthosterol −6.9 Kcal/mol
Ergsterol −6.5 Kcal/mol
Zymosterol −6.1 Kcal/mol
Parkeol −6.5 Kcal/mol

Experimental results indicate that many other cyclopentanoperhy drophenanthrene compounds also exhibit significant preventing or inhibiting effect to proteins accumulation in the inner lens. Cyclopentanoperhy drophenanthrene compounds of the present invention refers to a compound containing cyclopentanoperhy drophenanthrene skeleton structure, including its derivatives. After the two crystal protein mutant plasmid aA-crystallin-Y118D and aB-crystallin-R120G were introduced into cells, we investigated the lanosterol and its derivatives as well as cholesterol (Cholesterol) for their effects on intracellular accumulation of crystal protein. Not all cyclopentanoperhy drophenanthrene compound have the same effect. With the same experimental conditions as in FIG. 1, 10 μM cholesterol cannot effectively dissolve the intracellular accumulation of the protein crystal. The results show that parkeol, zymosterol, ergosterol, lanosterol, β-cholestanol, 5a-cholest-7-en-3β-ol significantly dissolve accumulated crystals protein.

Although the invention has been described with reference to the presently preferred embodiment, it should be understood that various modifications can be made without departing from the spirit of the invention.

SUPPLEMENTARY TABLE S1
Variant prioritization pipeline after exome sequencing

| | III-2 | IV-1 | IV-2 | IV-3 | |
|---|---|---|---|---|---|
| | (carrier | (affected | (affected | (affected | Combine |
| Filters | mother) | daughter) | son) | son) | samples |
| Total | 69,300 | 68,563 | 67,799 | 68,112 | — |
| variations | | | | | |
| Missense, | 7,808 | 7,662 | 7,445 | 7,528 | — |
| Nonsense, | | | | | |
| Read through | | | | | |
| and Splice | | | | | |
| site | | | | | |
| Homozygous | 5,078 | 2,830 | 2,871 | 2,853 | 120 |
| in affected | | | | | |
| child and | | | | | |
| heterozygous | | | | | |
| in carrier | | | | | |
| mother | | | | | |
| Not in | 1,140 | 69 | 94 | 94 | 6 |
| dbSNP | | | | | |
| Not in 1000 | 1,136 | 61 | 85 | 87 | 6 |
| Genomes | | | | | |
| Project | | | | | |
| Predicted | 228 | 10 | 13 | 14 | 2 (LSS, |
| damaging | | | | | WDR75) |

Supplementary Table S2. Primers using for
the construction of crystallin mutants.

| Gene | Primer (5′-3′) |
|---|---|
| αA-R116C-For | TTCCCGTGAGTTCCACTGCCGCTACCGCCTGCCGT |
| | CGCTGC |
| αA-R116C-Rev | CGGCAGGCGGTAGCGGCAGTGGAACTCACGGG |
| αA-R116H-For | TTCCCGTGAGTTCCACCACCGCTACCGCCTGCCGT |
| | CGCCAC |
| αA-R116H-Rev | CGGCAGGCGGTAGCGGTGGTGGAACTCACGGG |
| αA-Y118D-For | GAGTTCCACCGCCGCGACCGCCTGCCGTCCAACGT |
| | TACGAC |
| αA-Y118D-Rev | |
| αB-R120G-For | CAGGGAGTTCCACGGGAAATACCGGATAGGGGG |
| αB-R120G-Rev | GGATCCGGTATTTCCCGTGGAACTCCCT |
| βB2-V187E-For | AGGTGCAGTCCGAGCGCCGTATGTGGAG |
| βB2-V187E-Rev | ATACGGCGCTCGGACTGCACCT |
| βB2-V187M-For | AGGTGCAGTCCATGCGCCGTATGTGATG |
| βB2-V187M-Rev | ATACGGCGCTCGGACTGCACCT |
| βB2-R188H-For | TGCAGTCCGTGCACCGTATCCCGCCAC |
| βB2-R188H-Rev | GGATACGGTGCACGGACTGCA |
| γC-G129C-For | CACGTGCTGGAGTGCTGCTGGGCTGC |
| γC-G129C-Rev | CAGCAGCACTCCAGCACGTG |
| γD-W43R-For | GTGGACAGCGGCTGCCGGATGCTCTATGAGCTGGC |
| | GG |
| γD-W43R-Rev | GCTCATAGAGCATCCGGCAGCCGCTGTCCAC |

### REFERENCES

1 Pascolini, D. & Mariotti, S. P. Global estimates of visual impairment: 2010. Br J Ophthalmol 96, 614-618, doi:10.1136/bjophthalmol-2011-300539 (2012).
2 Bloemendal, H. et al. Ageing and vision: structure, stability and function of lens crystallins. Prog. Biophys. Mol. Biol. 86, 407-485 (2004).
3 Petrash, J. M. Aging and age-related diseases of the ocular lens and vitreous body. Invest Ophthalmol Vis Sci 54, ORSF54-59, doi:10.1167/iovs.13-12940 (2013).
4 Moreau, K. L. & King, J. A. Protein misfolding and aggregation in cataract disease and prospects for prevention. Trends Mol Med 18, 273-282, doi:S1471-4914(12)00039-1 [pii] 10.1016/j.molmed.2012.03.005 (2012).
5 Huff, M. W. & Telford, D. E. Lord of the rings—the mechanism for oxidosqualene:lanosterol cyclase becomes crystal clear. Trends Pharmacol Sci 26, 335-340, doi:S0165-6147(05)00127-6 [pii] 10.1016j.tips.2005.05.004 (2005).
6 Diehn, J. J., Diehn, M., Marmor, M. F. & Brown, P. O. Differential gene expression in anatomical compartments of the human eye. Genome Biol 6, R74, doi:gb-2005-6-9-r74 [pii] 10.1186/gb-2005-6-9-r74 (2005).
7 Ng, P. C. & Henikoff, S. Predicting deleterious amino acid substitutions. Genome Res 11, 863-874, doi:10.1101/gr.176601 (2001).
8 Thoma, R. et al. Insight into steroid scaffold formation from the structure of human oxidosqualene cyclase. Nature 432, 118-122, doi:10.1038/nature02993 (2004).
9 Cenedella, R. J. et al. Direct perturbation of lens membrane structure may contribute to cataracts caused by U18666A, an oxidosqualene cyclase inhibitor. J Lipid Res 45, 1232-1241, doi:10.1194/j1r.M300469-JLR200 M300469-JLR200 [pii] (2004).
10 Bloch, K. The biological synthesis of cholesterol. Science 150, 19-28 (1965).
11 Cardozo, T., Totrov, M. & Abagyan, R. Homology modeling by the ICM method. Proteins 23, 403-414, doi:10.1002/prot.340230314 (1995).
12 Abagyan, R. & Argos, P. Optimal protocol and trajectory visualization for conformational searches of peptides and proteins. J Mol Biol 225, 519-532, doi:0022-2836(92)90936-E [pii] (1992).
13 Xu, J. et al. The congenital cataract-linked A2V mutation impairs tetramer formation and promotes aggregation of βB2-crystallin. PLoS ONE 7, e51200 (2012).
14 Wang, B. et al. A novel CRYGD mutation (p.Trp43Arg) causing autosomal dominant congenital cataract in a Chinese family. Hum. Mutat. 32, E1939-E1947, doi:10.1002/humu.21386 (2011).
15 Gu, F. et al. A novel mutation in AlphaA-crystallin (CRYAA) caused autosomal dominant congenital cataract in a large Chinese family. Hum. Mutat. 29, 769 (2008).
16 Li, X.-Q. et al. A novel mutation impairing the tertiary structure and stability of γC-crystallin (CRYGC) leads to cataract formation in humans and zebrafish lens. Hum. Mutat. 33, 391-401, doi:10.1002/humu.21648 (2012).
17 Bligh, E. G. & Dyer, W. J. A rapid method of total lipid extraction and purification. Canadian journal of biochemistry and physiology 37, 911-917 (1959).
18 Wang, S., Leng, X.-Y. & Yan, Y.-B. The benefits of being β-crystallin heteromers: βB1-crystallin protects βA3-crystallin against aggregation during co-refolding. Biochemistry 50, 10451-10461, doi:10.1021/bi201375p (2011).
19 Sun, T.-X., Das, B. K. & Liang, J. J. N. Conformational and functional differences between recombinant human lens αA- and αB-crystallin. J Biol Chem 272, 6220-6225, doi:10.1074/jbc.272.10.6220 (1997).
20 Bradford, M. M. A rapid and sensitive method for the quantitation of microgram quantities of protein utilizing the principle of protein-dye binding. Anal. Biochem. 72, 248-254 (1976).

What is claimed is:

1. An ophthalmic pharmaceutical composition for treating and/or preventing vision disorders that affect the normal structure of the eye in a subject having or at risk of developing a vision disorder that affects the normal structure of the lens in the eye comprising administering to such subject a composition comprising a pharmaceutically acceptable ophthalmic carrier and a pharmaceutically effective amount of a sterol having a basic structure represented by formula 1:
formula I having a structure of: 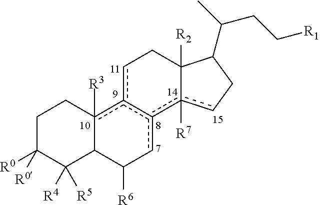
wherein:
R0 and R0′ is hydroxyl, —OSO3H, —OSO3-, —OCOCH3, —OPO3H, —OPO03-, or hydrogen, or R0 and R0′ together represent a carbonyl group;
R¹ is 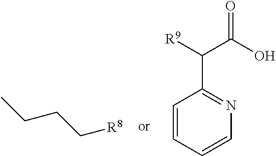
R², R³, R⁴, R⁵, R⁷ are each H or Me;
R⁶ is H or Me or OH or oxo (═O) or halide;
R⁸ is a linear or branched alkyl, aryl, alkene, alkyne, a substituted alkene, a substituted alkyl, a substituted alkyne, a substituted aryl, an alkyl halide, aikoxy such as an alcohol or an aryloxy, or an acetyl or ester group having from 2 to 6 carbon;
R¹ is at carbon 16 or 17, at least one of the dashed lines between carbons 7 and 8, carbons 8 and 9, carbons 9 and 10, carbons 9 and 11, carbons 8 and 14, or carbons 14 and 15 indicates a double bond, with the proviso that there be no adjacent double bonds on a ring or adjacent rings (e.g., if a double bond is present between carbons S and 9, no other double bonds are present in either of the two adjacent rings, or double bonds are not co-present between carbons 8 and 14 and carbons 14 and 15), and/or R³ is H if a double bond is present between carbons 9 and 10 and/or R⁷ is H if a double bond is present between carbons 8 and 14 or carbons 14 and 15; and a prodrug or pharmaceutically acceptable salt thereof.

2. The ophthalmic pharmaceutical composition of claim 1, wherein the sterol has a basic structure represented by formula IA: 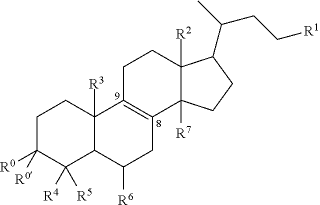
wherein:
R0 and R0′ is hydroxyl, —OSO3H, —OSO3-, —OCOCH3, —OPO3H, —OPO3-, or hydrogen, or R0 and R0′ together represent a carbonyl group;
R¹ is a linear or branched alkyl, aryl, alkene, alkyne, a substituted alkene, a substituted alkyl, a. substituted alkyne, a substituted aryl, an alkyl halide, alkoxy such as an alcohol or an aryloxy, or an acetyl or ester group having from 2 to 6 carbon;
R², R³, R⁴, R⁵, R⁷ are each H or Me; and
R⁶ is H or Me or OH or oxo (═O) or halide.

3. The ophthalmic pharmaceutical composition of claim 1, wherein said sterol is a cholesterol intermediate in the cholesterol biosynthesis selected from parkeol, zymosterol, ergosterol and lanosterol.

4. The ophthalmic pharmaceutical composition of claim 1, wherein said vision disorder affects the structure of the lens as to cause vision dysfunction.

5. The ophthalmic pharmaceutical composition of claim 1, wherein said vision disorder affects the clarity and/or rigidity of the lens of the eye.

6. The ophthalmic pharmaceutical composition of claim 1, wherein said vision disorder is a cataract, presbyopia nuclear sclerosis, or a retinal degenerative disorder selected from Refsum disease, Smith-Lemli-Opitz syndrome (SLOS) and Schnyder crystalline corneal dystrophy (SCCD), abetalipoproteinemia and familial hypobetalipoproteinemia.

7. The ophthalmic pharmaceutical composition of claim 1, wherein said sterol inhibits crystallin protein aggregation.

8. The ophthalmic pharmaceutical composition of claim 1, which is an ophthalmic solution, ophthalmic ointment, ophthalmic wash, intraocular infusion solution, wash for anterior chamber, internal medicine, injection, or preservative for extracted cornea.

9. The ophthalmic pharmaceutical composition of claim 1, wherein the pharmaceutically acceptable ophthalmic carrier is cyclodextrin.

10. The ophthalmic pharmaceutical composition of claim 1, wherein said composition further comprises a preservative.

11. A method for treating and/or preventing vision disorders that affect the normal structure of the eye in a subject having or at risk of developing a vision disorder that affects the normal structure of the lens in the eye comprising administering to such subject a composition comprising a pharmaceutically acceptable ophthalmic carrier and a pharmaceutically effective amount of a sterol having a basic structure represented by formula I: 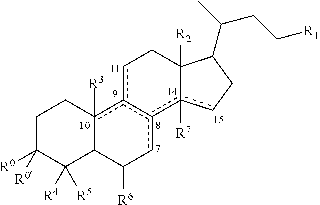
wherein:
R0 and R0′ is hydroxyl, —OSO3H, —OSO3-, —OCOCH3, —OPO3H, —OPO3-, or hydrogen, or R0 and R0′ together represent a carbonyl group;
R¹ is a linear or branched alkyl, aryl, alkene, alkyne, a substituted alkene, a substituted alkyl, a substituted alkyne, a substituted aryl, an alkyl halide, alkoxy such as an alcohol or an aryloxy, or an acetyl or ester group having from 2 to 6 carbon;
R² R³, R⁴, R⁵, R⁷ are each H or Me;
R⁶ is H or Me or OH or oxo (═O) or halide;
At least one of the dashed lines between carbons 7 and 8, carbons 8 and 9, carbons 9 and 10, carbons 9 and 11, carbons 8 and 14, or carbons 14 and 15 indicates a double bond, with the proviso that there be no adjacent double bonds on a ring or adjacent rings (e.g., if a double bond is present between carbons 8 and 9, no other double bonds are present in either of the two adjacent rings, or double bonds are not co-present between carbons 8 and 14 and carbons 14 and 15), and/or R³ is H if a double bond is present between carbons 9 and 10 and/or R⁷ is H if a double bond is present between carbons 8 and 14 or carbons 14 and 15.

12. The method of claim 11, wherein said vision disorder is selected from the group consisting of cataracts, nuclear sclerosis and presbyopia.

13. The method of claim 11, wherein said subject is selected from the group consisting of amphibians, reptiles, avians and mammals.

14. The method of claim 13, wherein said mammal is selected from the group consisting of rodents, cats, dogs, pigs, horses and humans.

15. The method of claim 13, wherein said mammal is a human.

16. The method of claim 11, wherein said composition is an ophthalmic solution, ophthalmic ointment, ophthalmic wash, intraocular infusion solution, wash for anterior chamber, internal medicine, injection, or preservative for extracted cornea.

17. The method of claim 11, wherein said pharmaceutically acceptable ophthalmic carrier is cyclodextrin.

18. The method of claim 11, wherein said composition further comprises a preservative.

19. A kit for treating and/or preventing vision disorders that affect the normal structure of the eye in a subject having or at risk of developing a vision disorder that affects the normal structure of the lens in the eye comprising a kit comprising a formulation of a pharmaceutically effective amount of a sterol having a basic structure represented by formula 1: 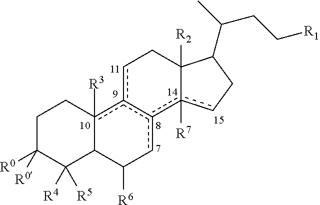
wherein:
R0 and R0′ is hydroxyl, —OSO3H, —OSO3-, —OCOCH3, —OPO3H, —OPO3-, or hydrogen, or R0 and R0′ together represent a carbonyl group;
R¹ is a linear or branched alkyl, aryl, alkene, alkyne, a substituted alkene, a substituted alkyl, a substituted alkyne, a substituted aryl, an alkyl halide, alkoxy such as an alcohol or an aryloxy, or an acetyl or ester group having from 2 to 6 carbon;
R², R³, R⁴, R⁵, R⁷ are each H or Me;
R⁶ is H or Me or OH or oxo (═O) or halide;
At least one of the dashed lines between carbons 7 and 8, carbons 8 and 9, carbons 9 and 10, carbons 9 and 11, carbons 8 and 14, or carbons 14 and 15 indicates a double bond, with the proviso that there be no adjacent double bonds on a ring or adjacent rings (e.g., if a double bond. is present between carbons 8 and 9, no other double bonds are present in either of the two adjacent rings, or double bonds are not co-present between carbons 8 and 14 and carbons 14 and 15), and/or R³ is H if a double bond is present between carbons 9 and 10 and/or R⁷ is H if a double bond is present between carbons 8 and 14 or carbons 14 and 15;
and a pharmaceutically acceptable carrier in a pharmaceutically acceptable carrier and instructions for administering said formulation such that said administration treats and/or prevents said vision disorder.
